(19) United States
(12) Patent Application Publication
(10) Pub. No.: US 2005/0261477 A1
(43) Pub. Date: Nov. 24, 2005

(54) Pharmaceutical compositions and medical treatments comprising notch ligand proteins

(76) Inventors: Brian Robert Champion, Cambridge (GB); Andrew Christopher Lennard, Cambridge (GB); Grahame James McKenzie, Cambridge (GB); Tamara Tugal, Cambridge (GB)

Correspondence Address: FROMMER LAWRENCE & HAUG, 745 FIFTH AVENUE- 10TH FL., NEW YORK, NY, 10151, US

(21) Appl. No.: 11/078,735

(22) Filed: Mar. 10, 2005

Related U.S. Application Data
(63) Continuation-in-part of application No. PCT/GB03/03908, filed on Sep. 9, 2003.

(30) Foreign Application Priority Data
Sep. 10, 2002 (GB) 0220912.0
Sep. 10, 2002 (GB) 0220913.8
Jan. 7, 2003 (GB) 0300234.2
Nov. 13, 2002 (WO) PCT/GB02/05137
Nov. 13, 2002 (WO) PCT/GB02/05133
Apr. 4, 2003 (WO) PCT/GB03/01525
Aug. 1, 2003 (WO) PCT/GB03/03285

(51) Int. Cl. C07K 14/705; C07H 21/04; C12P 21/06; C12N 15/09

(52) U.S. Cl. 530/350; 536/23.5; 435/69.1; 435/320.1; 435/325

(57) ABSTRACT

Provided is a Notch ligand protein or polypeptide comprising a Notch ligand DSL domain; 1 to 5 (but no more than 5) Notch ligand EGF repeat domains; optionally, all or part of a Notch ligand N-terminal domain; and optionally, one or more heterologous amino acid sequences. Also provided is a homogeneous or heterogeneous multimer of the protein or polypeptide and a polynucleotide encoding the Notch ligand protein or polypeptide. An immune response can be modified in a subject by administering the Notch ligand protein or polypeptide to the subject.

### CROSS REFERENCE TO RELATED APPLICATIONS

This application is a continuation-in-part of International Application No. PCT/GB2003/003908, filed on Sep. 9, 2003, published as WO 2004/024764 on Mar. 25, 2004, and claiming priority to International Application Nos. PCT/GB2002/005133 and PCT/GB2002/005137, both filed Nov. 13, 2002, GB Application Serial Nos. 0220912.0 and GB 0220913.8, both filed Sep. 10, 2002, PCT/GB2003/003285, filed Aug. 1, 2003, PCT/GB2003/001525, filed Apr. 4, 2003, and GB 0300234.2, filed Jan. 7, 2003.

Reference is made to U.S. application Ser. No. 09/310,685, filed May 4, 1999; Ser. No. 09/870,902, filed May 31, 2001; Ser. No. 10/013,310, filed Dec. 7, 2001; Ser. No. 10/147,354, filed May 16, 2002; Ser. No. 10/357,321, filed Feb. 3, 2002; Ser. No. 10/682,230, filed Oct. 9, 2003; Ser. No. 10/720,896, filed Nov. 24, 2003; Ser. Nos. 10/763,362, 10/764,415 and 10/765,727, all filed Jan. 23, 2004; Ser. No. 10/812,144, filed Mar. 29, 2004; Ser. Nos. 10/845,834 and 10/846,989, both filed May 14, 2004; Ser. No. 10/877,563, filed Jun. 25, 2004; Ser. No. 10/899,422, filed Jul. 26, 2004; Ser. No. 10/958,784, filed Oct. 5, 2004; Ser. No. 11/050,328, filed Feb. 3, 2005; and Ser. No. 11/058,066, filed Feb. 14, 2005. Reference is also made to the U.S. non-provisional application entitled, “Immunotherapy Using Modulators of Notch Signalling”, filed Mar. 3, 2005, attorney docket number 674525-2018.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to the modulation of Notch signalling, and preferably the modulation of immune function.

### BACKGROUND OF THE INVENTION

International Patent Publication No WO 98/20142 describes how manipulation of the Notch signalling pathway can be used in immunotherapy and in the prevention and/or treatment of T-cell mediated diseases. In particular, allergy, autoimmunity, graft rejection, tumour induced aberrations to the T-cell system and infectious diseases caused, for example, by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara, may be targeted.

It has also been shown that it is possible to generate a class of regulatory T cells which are able to transmit antigen-specific tolerance to other T cells, a process termed infectious tolerance (WO98/20142). The functional activity of these cells can be mimicked by over-expression of a Notch ligand protein on their cell surfaces or on the surface of antigen presenting cells. In particular, regulatory T cells can be generated by over-expression of a member of the Delta or Serrate family of Notch ligand proteins.

A description of the Notch signalling pathway and conditions affected by it may be found, for example, in our published PCT Applications as follows:
PCT/GB97/03058 (filed on 6 Nov. 1997 and published as WO 98/20142; claiming priority from GB 9623236.8 filed on 7 Nov. 1996, GB 9715674.9 filed on 24 Jul. 1997 and GB 9719350.2 filed on 11 Sep. 1997);
PCT/GB99/04233 (filed on 15 Dec. 1999 and published as WO 00/36089; claiming priority from GB 9827604.1 filed on 15 Dec. 1999);
PCT/GB00/04391 (filed on 17 Nov. 2000 and published as WO 0135990; claiming priority from GB 9927328.6 filed on 18 Nov. 1999);
PCT/GB01/03503 (filed on 3 Aug. 2001 and published as WO 02/12890; claiming priority from GB 0019242.7 filed on 4 Aug. 2000);
PCT/GB02/02438 (filed on 24 May 2002 and published as WO 02/096952; claiming priority from GB 0112818.0 filed on 25 May 2001);
PCT/GB02/03381 (filed on 25 July 2002 and published as WO 03/012111; claiming priority from GB 0118155.1 filed on 25 Jul. 2001);
PCT/GB02/03397 (filed on 25 Jul. 2002 and published as WO 03/012441; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);
PCT/GB02/03426 (filed on 25 Jul. 2002 and published as WO 03/011317; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);
PCT/GB02/04390 (filed on 27 Sep. 2002 and published as WO 03/029293; claiming priority from GB 0123379.0 filed on 28 Sep. 2001);
PCT/GB02/05137 (filed on 13 Nov. 2002 and published as WO 03/041735; claiming priority from GB 0127267.3 filed on 14 Nov. 2001, PCT/GB02/03426 filed on 25 Jul. 2002, GB 0220849.4 filed on 7 Sep. 2002, GB 0220913.8 filed on 10 Sep. 2002 and PCT/GB02/004390 filed on 27 Sep. 2002);
PCT/GB02/05133 (filed on 13 Nov. 2002 and published as WO 03/042246; claiming priority from GB 0127271.5 filed on 14 Nov. 2001 and GB 0220913.8 filed on 10 Sep. 2002).

Each of PCT/GB97/03058 (WO 98/20142), PCT/GB99/04233 (WO 00/36089), PCT/GB00/04391 (WO 0135990), PCT/GB01/03503 (WO 02/12890), PCT/GB02/02438 (WO 02/096952), PCT/GB02/03381 (WO 03/012111), PCT/GB02/03397 (WO 03/012441), PCT/GB02/03426 (WO 03/011317), PCT/GB02/04390 (WO 03/029293), PCT/GB02/05137 (WO 03/041735) and PCT/GB02/05133 (WO 03/042246) is hereby incorporated herein by reference

Reference is made also to Hoyne G. F. et al (1999) Int Arch Allergy Immunol 18:122-124; Hoyne et al. (2000) Immunology 100:281-288; Hoyne G. F. et al (2000) Intl Immunol 12:177-185; Hoyne, G. et al. (2001) Immunological Reviews 182:215-227; each of which is also incorporated herein by reference.

The present invention seeks to provide further methods, constructs and compositions for modulating the Notch signalling pathway.

### SUMMARY OF THE INVENTION

According to a first aspect of the invention there is provided a method for modifying an immune response by administering a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or by administering a polynucleotide coding for such a Notch ligand protein or polypeptide.

According to a further aspect of the invention there is provided a method for reducing an immune response by administering a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or by administering a polynucleotide coding for such a Notch ligand protein or polypeptide.

According to a further aspect of the invention there is provided a method for increasing immune tolerance by administering a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and

iv) optionally one or more heterologous amino acid sequences; or by administering a polynucleotide coding for such a Notch ligand protein or polypeptide.

According to a further aspect of the invention there is provided a method for modifying T cell activity by administering a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or by administering a polynucleotide coding for such a Notch ligand protein or polypeptide.

According to a further aspect of the invention there is provided a method for reducing helper (T_{H}) or cytotoxic (T_{C}) T-cell activity by administering a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or by administering a polynucleotide coding for such a Notch ligand protein or polypeptide.

According to a further aspect of the invention there is provided a method for increasing activity of regulatory T cells by administering a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or by administering a polynucleotide coding for such a Notch ligand protein or polypeptide. Suitably the regulatory T cells are Tr1 regulatory T-cells.

Suitably the protein, polypeptide or polynucleotide is administered to a patient in vivo.

Alternatively the protein, polypeptide or polynucleotide may be administered to cells from a patient ex vivo. Suitably the cells may then be administered to a patient after administration of the protein, polypeptide or polynucleotide.

According to a further aspect of the invention there is provided a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or a polynucleotide coding for such a Notch ligand protein or polypeptide,for use to treat disease.

According to a further aspect of the invention there is provided the use of a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences;

or a polynucleotide coding for such a Notch ligand protein or polypeptide, in the manufacture of a medicament for reduction of an immune response.

According to a further aspect of the invention there is provided the use of a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for modification of an immune response.

According to a further aspect of the invention there is provided the use of a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for reduction of an immune response.

According to a further aspect of the invention there is provided the use of a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for increasing immune tolerance.

According to a further aspect of the invention there is provided the use of a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for modification of T-cell activity.

According to a further aspect of the invention there is provided the use of a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for reduction of helper (T_{H}) or cytotoxic (T_{C}) T-cell activity.

According to a further aspect of the invention there is provided the use of a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for increasing activity of regulatory T cells.

Suitably the Notch ligand protein or polypeptide consists essentially of the following components:
i) a Notch ligand DSL domain;
ii) one Notch ligand EGF domain;
iii) optionally all or part of a Notch ligand N-terminal domain; and
iv) optionally one or more heterologous amino acid sequences; or a polynucleotide which codes for such a Notch ligand protein or polypeptide.

Alternatively the Notch ligand protein or polypeptide consists essentially of the following components:
i) a Notch ligand DSL domain;
ii) two Notch ligand EGF domains;
iii) optionally all or part of a Notch ligand N-terminal domain; and
iv) optionally one or more heterologous amino acid sequences; or a polynucleotide which codes for such a Notch ligand protein or polypeptide.

Alternatively the Notch ligand protein or polypeptide consists essentially of the following components:
i) a Notch ligand DSL domain;
ii) three Notch ligand EGF domains;
iii) optionally all or part of a Notch ligand N-terminal domain; and
iv) optionally one or more heterologous amino acid sequences; or a polynucleotide sequence which codes for such a Notch ligand protein or polypeptide.

According to a further aspect of the invention there is provided a pharmaceutical composition comprising a Notch ligand protein or polypeptide consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences; or a polynucleotide coding for such a Notch ligand protein or polypeptide, optionally in combination with a pharmaceutically acceptable carrier.

According to a further aspect of the invention there is provided a Notch ligand protein or polypeptide which consists essentially of the following components:
i) a Notch ligand DSL domain;
ii) 1 to 5 (preferably 1 to 4, preferably 1 to 3) Notch ligand EGF domains;
iii) optionally all or part of a Notch ligand N-terminal domain; and
iv) optionally one or more heterologous amino acid sequences; or a polynucleotide which codes for such a Notch ligand protein or polypeptide.

According to a further aspect of the invention there is provided a Notch ligand protein or polypeptide which consists essentially of the following components:
i) a Notch ligand DSL domain;
ii) one Notch ligand EGF domain;
iii) optionally all or part of a Notch ligand N-terminal domain; and
iv) optionally one or more heterologous amino acid sequences; or a polynucleotide which codes for such a Notch ligand protein or polypeptide.

According to a further aspect of the invention there is provided a Notch ligand protein or polypeptide which consists essentially of the following components:
i) a Notch ligand DSL domain;
ii) two Notch ligand EGF domains;
iii) optionally all or part of a Notch ligand N-terminal domain; and
iv) optionally one or more heterologous amino acid sequences; or a polynucleotide which codes for such a Notch ligand protein or polypeptide.

According to a further aspect of the invention there is provided a Notch ligand protein or polypeptide which consists essentially of the following components:
i) a Notch ligand DSL domain;
ii) three Notch ligand EGF domains;
iii) optionally all or part of a Notch ligand N-terminal domain; and
iv) optionally one or more heterologous amino acid sequences; or a polynucleotide sequence which codes for such a Notch ligand protein or polypeptide.

According to a further aspect of the invention there is provided a multimeric Notch ligand protein or polypeptide comprising monomers consisting essentially of the following components:
i) a Notch ligand DSL domain;
ii) 1 to 5 (preferably 1 to 4, preferably 1 to 3) EGF repeat domains;
iii) optionally all or part of a Notch ligand N-terminal domain; and
iv) optionally one or more heterologous amino acid sequences;
wherein each monomer may be the same or different. Where each monomer is the same as each other monomer, the multimeric Notch ligand protein or polypeptide is referred to as “homogenous.” Where the monomers are different from one another, the multimer is referred to as “heterogenous.”

According to a further aspect of the invention there is provided a multimeric Notch ligand protein or polypeptide comprising monomers comprising:
i) a Notch ligand DSL domain;
ii) 1 to 5 (but no more than 5) EGF repeat domains;
iii) optionally all or part of a Notch ligand N-terminal domain; and
iv) optionally one or more heterologous amino acid sequences;
   wherein each monomer may be the same or different.

According to a further aspect of the invention there is provided a Notch ligand protein or polypeptide consisting essentially of the following components:
i) a Notch ligand DSL domain;
ii) 1 to 5 EGF repeat domains;
iii) optionally all or part of a Notch ligand N-terminal domain; and
iv) optionally one or more heterologous amino acid sequences;
or a multimer of such a protein or polypeptide (wherein each monomer may be the same or different);
   or a polynucleotide coding for such a Notch ligand protein or polypeptide; for use in the treatment of disease.

According to a further aspect of the invention there is provided a Notch ligand protein or polypeptide comprising:
i) a Notch ligand DSL domain;
ii) 1 to 5 (but no more than 5) EGF repeat domains;
iii) optionally all or part of a Notch ligand N-terminal domain; and
iv) optionally one or more heterologous amino acid sequences;
   or a multimer of such a protein or polypeptide (wherein each monomer may be the same or different); or a polynucleotide coding for such a Notch ligand protein or polypeptide; for use in the treatment of disease.

According to a further aspect of the invention there is provided a method of therapeutically modulating Notch signalling by administering a Notch ligand protein or polypeptide consisting essentially of the following components:
i) a Notch ligand DSL domain;
ii) 1 to 5 EGF repeat domains;
iii) optionally all or part of a Notch ligand N-terminal domain; and
iv) optionally one or more heterologous amino acid sequences;
   or a multimer of such a protein or polypeptide (wherein each monomer may be the same or different); or a polynucleotide coding for such a Notch ligand protein or polypeptide.

According to a further aspect of the invention there is provided a method of therapeutically modulating Notch signalling by administering a Notch ligand protein or polypeptide comprising:
i) a Notch ligand DSL domain;
ii) 1 to 5 (but no more than 5) EGF repeat domains;
iii) optionally all or part of a Notch ligand N-terminal domain; and
iv) optionally one or more heterologous amino acid sequences;
   or a multimer of such a protein or polypeptide (wherein each monomer may be the same or different); or a polynucleotide coding for such a Notch ligand protein or polypeptide.

According to a further aspect of the invention there is provided a vector comprising a polynucleotide coding for a Notch ligand protein or polypeptide as described above.

According to a further aspect of the invention there is provided a host cell transformed or transfected with such a vector.

According to a further aspect of the invention there is provided a cell displaying a Notch ligand protein or polypeptide as described above on its surface and/or transfected with a polynucleotide coding for such a protein or polypeptide.

Suitably the protein or polypeptide is not bound to a cell. Alternatively, the protein or polypeptide may be cell-associated.

In one embodiment the Notch ligand elements of the protein or polypeptide may be fused to a heterologous amino acid sequence, such as a sequence corresponding to all or part of an immunoglobulin F_{c} segment. In one embodiment, particularly where the Notch ligand protein or polypeptide comprises two EGF repeat domains, the heterologous amino acid sequence is not a TSST sequence, or is not a superantigen sequence.

Preferably the protein or polypeptide comprises at least part of a mammalian, preferably human, Notch ligand sequence.

Suitably the protein or polypeptide comprises Notch ligand domains from Delta, Serrate or Jagged or domains having at least 30% (preferably at least 50%, at least 70%, at least 90% or at least 95%) amino acid sequence similarity or identity thereto.

Suitably the protein or polypeptide comprises Notch ligand domains from Deltal, Delta 3, Delta 4, Jagged 1 or Jagged 2 or domains having at least 30% (preferably at least 50%, at least 70%, at least 90% or at least 95%) amino acid sequence similarity or identity thereto.

Suitably the protein or polypeptide activates a Notch receptor (preferably human Notch1, Notch2, Notch3 or Notch4). Alternatively it may inhibit a Notch receptor.

Suitably the protein or polypeptide is a Notch signalling agonist or partial agonist. Alternatively it may be a Notch signalling antagonist.

According to a further aspect of the invention there is provided a polynucleotide coding for a protein or polypeptide as described above. According to further aspects of the invention there are provided a vector comprising such a polynucleotide and a host cell transformed or transfected with such a vector.

According to a further aspect of the invention there is provided a cell displaying a Notch ligand protein or polypeptide as described above on its surface and/or transfected with a polynucleotide coding for such a protein or polypeptide.

Suitably such a cell may further display at least one antigen or antigenic determinant, for example a tumour antigen or antigenic determinant.

Suitably the modulation of the immune system comprises treatment of asthma, allergy, graft rejection, autoimmunity, cancer, tumour induced aberrations to the immune system or infectious disease.

In one embodiment the modulator of the Notch signalling pathway may comprise a fusion protein comprising domains from a Notch ligand extracellular domain and an immunoglobulin F_{c} segment (e.g. IgG1 Fc or IgG4 Fc) or a polynucleotide coding for such a fusion protein. Methods suitable for preparation of such fusion proteins are described, for example in Example 2 of WO 98/20142. IgG fusion proteins may be prepared as well known in the art, for example, as described in U.S. Pat. No. 5,428,130 (Genentech).

Suitably, in one embodiment of the invention, the Notch ligand protein or polypeptide may be bound to a support, preferably a particulate support. Thus, according to a further aspect of the invention there is provided a particle comprising a Notch ligand protein or polypeptide as described above bound to a particulate support matrix. In one embodiment the particulate support matrix may be a bead. The bead may be, for example, a magnetic bead (e.g. as available under the trade name “Dynal”) or a polymeric bead such as a Sepharose bead.

According to a further aspect of the invention there is provided a particle wherein a plurality of Notch ligand proteins or polypeptides as described above are bound to a particulate support matrix.

According to a further aspect of the invention there is provided a method for reducing TNFα expression by administering a protein, polypeptide or polynucleotide as described above.

According to a further aspect of the invention there is provided a method for increasing IL-10 expression by administering a protein, polypeptide or polynucleotide as described above.

According to a further aspect of the invention there is provided a method for reducing IL-5 expression by administering a protein, polypeptide or polynucleotide as described above.

According to a further aspect of the invention there is provided a method for reducing IL-13 expression by administering a protein, polypeptide or polynucleotide as described above.

Suitably the protein, polypeptide or polynucleotide modifies cytokine expression in leukocytes (such as lymphocytes or macrophages), fibroblasts or epithelial cells or their progenitors or tissue-specific derivatives.

According to a further aspect of the invention there is provided a method for generating an immune modulatory cytokine profile with increased IL-10 expression and reduced TNFα expression by administering a protein, polypeptide or polynucleotide as described above.

According to a further aspect of the invention there is provided a method for generating an immune modulatory cytokine profile with increased IL-10 expression and reduced IL-5 expression by administering a protein, polypeptide or polynucleotide as described above.

According to a further aspect of the invention there is provided a method for generating an immune modulatory cytokine profile with increased IL-10 expression and reduced IL-13 expression by administering a protein, polypeptide or polynucleotide as described above.

According to a further aspect of the invention there is provided a method for generating an immune modulatory cytokine profile with reduced IL-5, IL-13 and TNFα expression by administering a protein, polypeptide or polynucleotide as described above.

According to a further aspect of the invention there is provided a method for generating an immune modulatory cytokine profile with reduced IL-2, IFN-γ, IL-5, IL-13 and TNFα expression by administering a protein, polypeptide or polynucleotide as described above.

Suitably the cytokine profile also exhibits increased IL-10 expression.

According to a further aspect of the invention there is provided a method for reducing a TH2 immune response by administering a protein, polypeptide or polynucleotide as described above.

According to a further aspect of the invention there is provided a method for reducing a TH1 immune response by administering a protein, polypeptide or polynucleotide as described above.

According to a further aspect of the invention there is provided a method for treating inflammation or an inflammatory condition by administering a protein, polypeptide or polynucleotide as described above.

According to a further aspect of the invention there is provided a method for treating inflammation or an inflammatory or autoimmune condition by administering a protein, polypeptide or polynucleotide as described above to reduce TNFα expression.

In one embodiment of the invention the protein, polypeptide or polynucleotide as described above is administered to a patient in vivo. Alternatively the modulator of Notch signalling may be administered to a cell ex-vivo, after which the cell may be administered to a patient.

According to a further aspect of the invention there is provided a method for the treatment of a disease associated with excessive IL-5 production by administering a protein, polypeptide or polynucleotide as described above.

According to a further aspect of the invention there is provided a method for the treatment of a disease associated with excessive IL-13 production by administering a protein, polypeptide or polynucleotide as described above.

Preferably a modulator of Notch signalling will be in a multimerised form. The number of monomers in the multimer may be at least 2, at least 4, at least 5, at least 10, at least 20, at least 30, at least 40, at least 50 or more, for example at least about 3 to 100 or more, for example at least about 10 to 50 or more.

Modulators of Notch signalling in the form of Notch ligand proteins/polypeptides coupled to polymer supports are described in Lorantis Ltd's co-pending PCT application No PCT/GB2003/003285 (filed on 1 Aug. 2003 claiming priority from GB 0218068.5), the text of which is herein incorporated by reference (e.g. see in particular Example 5 therein disclosing a dextran conjugate).

In another embodiment, modulators of Notch signalling in the form of Notch ligand proteins/polypeptides coupled to particulate supports such as beads are described in WO 03/011317 (Lorantis) and in Lorantis' co-pending PCT application PCT/GB2003/001525 (filed on 4 Apr. 2003), the texts of which are hereby incorporated by reference (e.g. see in particular Examples 17, 18, 19 of PCT/GB2003/001525).

The term “which consists essentially of” or “consisting essentially of” as used herein means that the construct includes the sequences and domains identified but is substantially free of other sequences or domains, and in particular is substantially free of any other Notch or Notch ligand sequences or domains.

For avoidance of doubt the term “comprising” means that any additional feature or component may be present.

Due to their generally smaller size compared to naturally occurring Notch ligands, the constructs of the present invention provide for easier manufacturing and/or administration whilst still retaining effective biological activity.

### BRIEF DESCRIPTION OF THE DRAWINGS

Various preferred features and embodiments of the present invention will now be described in more detail by way of non-limiting example and with reference to the accompanying Figures, in which:

FIG. 1 shows a schematic representation of the Notch signalling pathway;

FIG. 2 shows schematic representations of the Notch ligands Jagged and Delta;

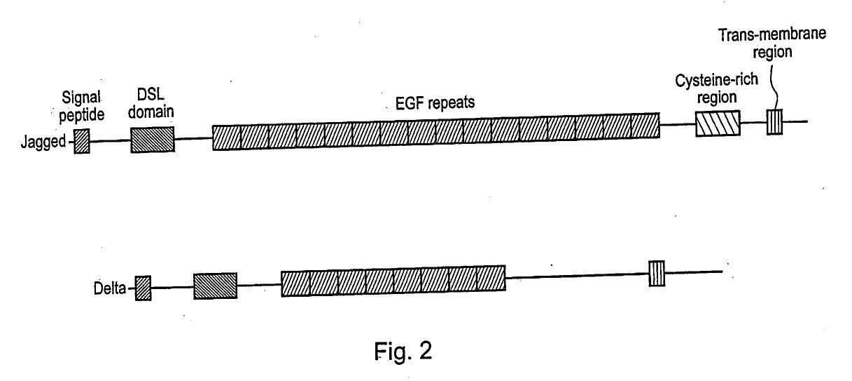

FIG. 3 shows aligned amino acid sequences of DSL domains from various Drosophila and mammalian Notch ligands; (SEQ ID NOs:1-16)

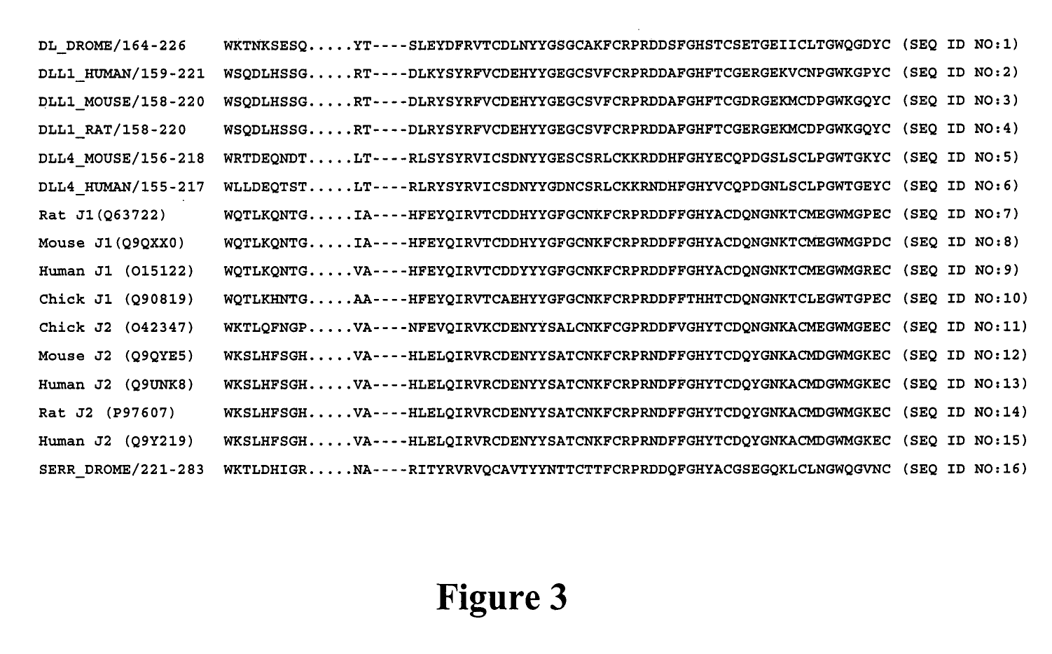

FIGS. 4A-4C show the amino acid sequences of human Delta-1 (4A; SEQ ID NO:17), Delta-3 (4B; SEQ ID NO:18) and Delta-4 (4C; SEQ ID NO:19);

FIGS. 5A and 5B show the amino acid sequences of human Jagged-1 (SEQ ID NO:20) and Jagged-2 (SEQ ID NO:21), respectively;

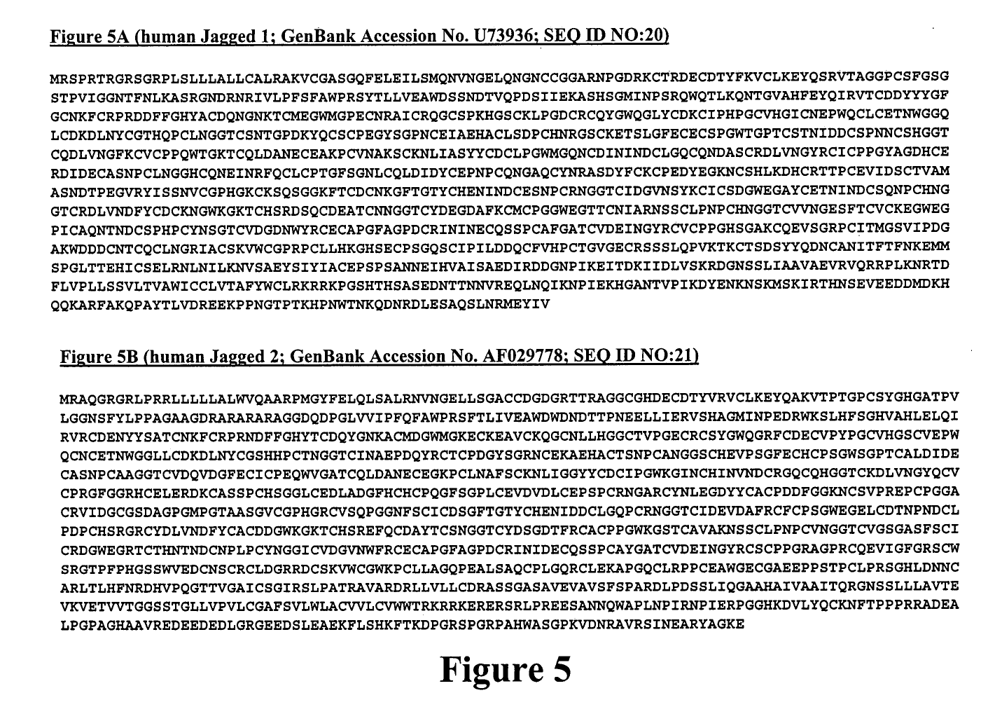

FIG. 6 shows schematic representations of Notch ligand protein and polypeptide constructs according to various embodiments of the invention;

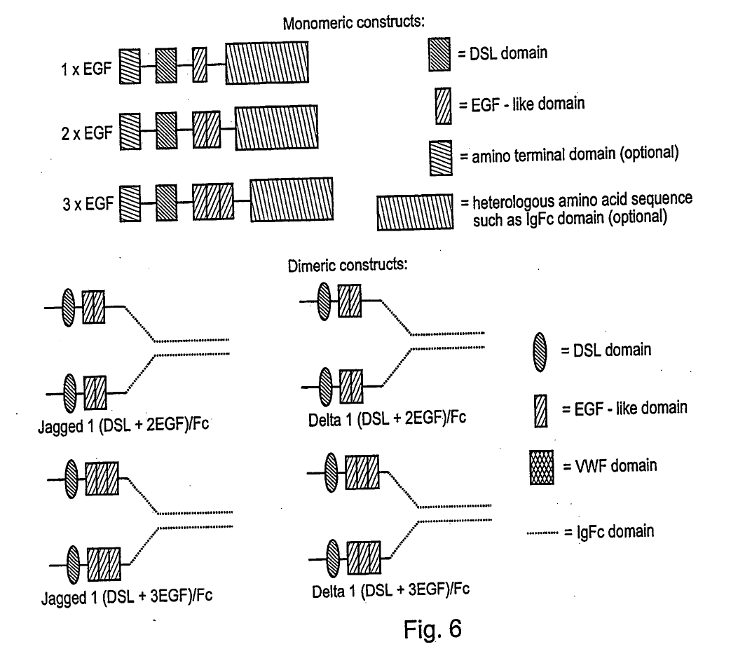

FIG. 7 is a Western blot showing results from Example 5;

FIG. 8 is a bar graph showing results from Example 7;

FIG. 9 is a bar graph showing results from Example 8;

FIG. 10 is a bar graph showing results from Examples 7 and 8;

FIG. 11 is a bar graph showing results from Example 9;

FIG. 12 is a line graph showing results from Example 10;

FIG. 13 shows results of SDS-PAGE and Western blot experiments from Example 12;

FIG. 14 shows SDS-PAGE experiments from Example 13(i); and

FIG. 15 shows the elution profile and SDS-PAGE results from Example 13(i).

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and J. E. Coligan, A. M. Kruisbeek, D. H. Margulies, E. M. Shevach and W. Strober (1992 and periodic supplements; Current Protocols in Immunology, John Wiley & Sons, New York, N.Y.). Each of these general texts is herein incorporated by reference.

The term “modulate” as used herein refers to a change or alteration in the biological activity of the Notch signalling pathway or a target signalling pathway thereof. Preferably, the term “modulator” as used herein refers to agonists of Notch signalling, i.e. compounds which stimulate or upregulate, at least to some extent, the normal biological activity of the Notch signalling pathway. Conveniently such agents may be referred to as upregulators or agonists.

Preferably the protein, polypeptide or polynucleotide is or codes for an agonist of Notch signalling, and suitably an agonist/activator of the Notch receptor (e.g. an agonist/activator of the human Notch1, Notch2, Notch3 and/or Notch4 receptor).

The skilled worker can readily determine whether a given agent is an agonist or antagonist of Notch signalling by testing the agent in an assay as well known in the art.

Agonist activity may suitably be determined by use of an agonist assay, for example a Notch signalling reporter assay as described in Examples 6 and 7 herein.

Antagonist activity may suitably be determined by use of an antagonist assay, for example as described in Example 10 herein.

In the present invention Notch signalling preferably means specific signalling, meaning that the signalling results substantially or at least predominantly from the Notch signalling pathway, and preferably from Notch/Notch ligand interaction, rather than any other significant interfering or competing cause, such as cytokine signalling. The Notch signalling pathway is described in more detail below.

Proteins or polypeptides may be in the form of the “mature” protein or may be a part of a larger protein such as a fusion protein or precursor. For example, it is often advantageous to include an additional (heterologous) amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Key targets for Notch-dependent transcriptional activation are genes of the Enhancer of split complex (E[spl]). Moreover these genes have been shown to be direct targets for binding by the Su(H) protein and to be transcriptionally activated in response to Notch signalling. By analogy with EBNA2, a viral coactivator protein that interacts with a mammalian Su(H) homologue CBF1 to convert it from a transcriptional repressor to a transcriptional activator, the Notch intracellular domain, perhaps in association with other proteins may combine with Su(H) to contribute an activation domain that allows Su(H) to activate the transcription of E(spl) as well as other target genes. It should also be noted that Su(H) is not required for all Notch-dependent decisions, indicating that Notch mediates some cell fate choices by associating with other DNA-binding transcription factors or be employing other mechanisms to transduce extracellular signals.

By a protein or polypeptide which is for Notch signalling activation we mean a molecule which is capable of activating Notch, the Notch signalling pathway or any one or more of the components of the Notch signalling pathway.

In one embodiment, the active agent may be a Notch ligand, or a polynucleotide encoding a Notch ligand. Notch ligands of use in the present invention include endogenous Notch ligands which are typically capable of binding to a Notch receptor polypeptide present in the membrane of a variety of mammalian cells, for example hemapoietic stem cells.

The term “Notch ligand” as used herein means an agent capable of interacting with a Notch receptor to cause a biological effect. The term as used herein therefore includes naturally occurring protein ligands such as Delta and Serrate/Jagged as well as antibodies to the Notch receptor, peptidomimetics and small molecules which have corresponding biological effects to the natural ligands. Preferably the Notch ligand interacts with the Notch receptor by binding.

Particular examples of mammalian Notch ligands identified to date include the Delta family, for example Delta or Delta-like 1 (Genbank Accession No. AF003522—Homo sapiens), Delta-3 (Genbank Accession No. AF084576—Rattus norvegicus) and Delta-like 3 (Mus musculus) (Genbank Accession No. NM_{—}016941—Homo sapiens) and U.S. Pat. No. 6121045 (Millennium), Delta-4 (Genbank Accession Nos. AB043894 and AF 253468—Homo sapiens) and the Serrate family, for example Serrate-1 and Serrate-2 (WO97/01571, WO96/27610 and WO92/19734), Jagged-1 (Genbank Accession No. U73936—Homo sapiens) and Jagged-2 (Genbank Accession No. AF029778—Homo sapiens), and LAG-2. Homology between family members is extensive.

By polypeptide for Notch signalling activation is also meant any polypeptides expressed as a result of Notch activation and any polypeptides involved in the expression of such polypeptides, or polynucleotides coding for such polypeptides.

Preferably when the inhibitor is a receptor or a nucleic acid sequence encoding a receptor, the receptor is activated. Thus, for example, when the agent is a nucleic acid sequence, the receptor is preferably constitutively active when expressed.

Any one or more of appropriate targets—such as an amino acid sequence and/or nucleotide sequence—may be used for identifying a compound capable of modulating the Notch signalling pathway and/or a targeting molecule in any of a variety of drug screening techniques. The target employed in such a test may be free in solution, affixed to a solid support, borne on a cell surface, or located intracellularly.

Techniques for drug screening may be based on the method described in Geysen, European Patent No. 0138855, published on Sep. 13, 1984. In summary, large numbers of different small peptide candidate modulators or targeting molecules are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators and targeting molecules.

This invention also contemplates the use of competitive drug screening assays in which neutralising antibodies capable of binding a target specifically compete with a test compound for binding to a target.

Techniques are well known in the art for the screening and development of agents such as antibodies, peptidomimetics and small organic molecules which are capable of binding to components of the Notch signalling pathway. These include the use of phage display systems for expressing signalling proteins, and using a culture of transfected E. coli or other microorganism to produce the proteins for binding studies of potential binding compounds (see, for example, G. Cesarini, FEBS Letters, 307(1):66-70 (July 1992); H. Gram et al., J. Immunol. Meth., 161:169-176 (1993); and C. Summer et al., Proc. Natl. Acad. Sci., USA, 89:3756-3760 (May 1992)). Further library and screening techniques are described, for example, in U.S. Pat. No. 6,281,344 (Phylos).

Within the definitions of “proteins” useful in the present invention, the specific amino acid residues may be modified in such a manner that the protein in question retains at least one of its endogenous functions, such modified proteins are referred to as “variants”. A variant protein can be modified by addition, deletion and/or substitution of at least one amino acid present in the naturally-occurring protein.

Typically, amino acid substitutions may be made, for example from 1, 2 or 3 to 10 or 20 substitutions provided that the modified sequence retains the required target activity or ability to modulate Notch signalling. Amino acid substitutions may include the use of non-naturally occurring analogues.

The protein used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the target or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| Symbol | 3-letter | Meaning | Codons |
|---|---|---|---|
| A | Ala | Alanine | GCT, GCC, GCA, GCG |
| B | Asp, | Aspartic, | GAT, GAC, AAT, AAC |
| | Asn | Asparagine | |
| C | Cys | Cysteine | TGT, TGC |
| D | Asp | Aspartic | GAT, GAC |
| E | Glu | Glutamic | GAA, GAG |
| F | Phe | Phenylalanine | TTT, TTC |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, CTC, CTA, CTG |
| M | Met | Methionine | ATG |
| N | Asn | Asparagine | AAT, AAC |
| P | Pro | Proline | CCT, CCC, CCA, CCG |
| Q | Gln | Glutamine | CAA, CAG |
| R | Arg | Arginine | CGT, CGC, CGA, CGG, AGA, AGG |
| S | Ser | Serine | TCT, TCC, TCA, TCG, AGT, AGC |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, | Glutamic, | GAA, GAG, CAA, CAG |
| | Gln | Glutamine | |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function. A peptide useful in the invention will at least have a target or signalling modulation capability. “Fragments” are also variants and the term typically refers to a selected region of the protein that is of interest in a binding assay and for which a binding partner is known or determinable. “Fragment” thus refers to an amino acid sequence that is a portion of a full-length polypeptide, preferably between about 8 and about 745 amino acids in length, preferably about 8 to about 300, more preferably about 8 to about 200 amino acids, and even more preferably about 10 to about 50 or 100 amino acids in length. “Peptide” refers to a short amino acid sequence that is 10 to 40 amino acids long, preferably 10 to 35 amino acids.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Variants of the nucleotide sequence may also be made. Such variants will preferably comprise codon optimised sequences. Codon optimisation is known in the art as a method of enhancing RNA stability and therefore gene expression. The redundancy of the genetic code means that several different codons may encode the same amino-acid. For example, leucine, arginine and serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms.

Where the active agent is a nucleotide sequences it may suitably be codon optimised for expression in mammalian cells. In a preferred embodiment, such sequences are optimised in their entirety.

“Polynucleotide” refers to a polymeric form of nucleotides of at least 10 bases in length and up to 10,000 bases or more, either ribonucleotides or deoxyribonucleotides or a modified form of either type of nucleotide. The term includes single and double stranded forms of DNA and also derivafised versions such as protein nucleic acid (PNA).

These may be constructed using standard recombinant DNA methodologies. The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Nucleic acid encoding the second region may likewise be provided in a similar vector system.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data are available or where it is desired to express a nucleic acid homologous or otherwise related to a known nucleic acid, exemplary nucleic acids can be characterised as those nucleotide sequences which hybridise to the nucleic acid sequences known in the art.

It will be understood by a skilled person that numerous different nucleotide sequences can encode the same protein used in the present invention as a result of the degeneracy of the genetic code. In addition, it is to be understood that skilled persons may, using routine techniques, make nucleotide substitutions that do not affect the protein encoded by the nucleotide sequence of the present invention to reflect the codon usage of any particular host organism in which the target protein or protein for Notch signalling modulation of the present invention is to be expressed.

In general, the terms “variant”, “homologue” or “derivative” in relation to the nucleotide sequence used in the present invention includes any substitution of, variation of, modification of, replacement of, deletion of or addition of one (or more) nucleic acid from or to the sequence providing the resultant nucleotide sequence codes for a target protein or protein for T cell signalling modulation.

As indicated above, with respect to sequence homology, preferably there is at least 75%, more preferably at least 85%, more preferably at least 90% homology to the reference sequences. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the reference sequences, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction (PCR) technologies.

Nucleotide sequences useful in the invention capable of selectively hybridising to the nucleotide sequences presented herein, or to their complement, will be generally at least 75%, preferably at least 85 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences of the invention will comprise regions homologous to the nucleotide sequence, preferably at least 80 or 90% and more preferably at least 95% homologous to the nucleotide sequence.

The term “selectively hybridizable” means that the nucleotide sequence used as a probe is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences which are not 100% homologous to the sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the target protein or protein for T cell signalling modulation encoded by the nucleotide sequences.

Sequence Homology, Similarity and Identity

As used herein, the term “homology” can be equated with “identity”. An homologous sequence will be taken to include an amino acid sequence which may be at least 75, 85 or 90% identical, preferably at least 95 or 98% identical. In particular, homology should typically be considered with respect to those regions of the sequence (such as amino acids at positions 51, 56 and 57) known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefor firstly requires the production of an optimal alignment, taking into consideration gap penalties. A suitable computer program for carrying out such an alignment is the GCG Wisconsin Bestfit package (University of Wisconsin, U.S.A.; Devereux). Examples of other software than can perform sequence comparisons include, but are not limited to, the BLAST package, FASTA (Atschul et al. (1990) J. Mol. Biol. 403-410 (Atschul)) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

The five BLAST programs, available from the U.S. National Institutes of Health National Center for Biotechnology Information, perform the following tasks:
blastp—compares an amino acid query sequence against a protein sequence database.
blastn—compares a nucleotide query sequence against a nucleotide sequence database.
blastx—compares the six-frame conceptual translation products of a nucleotide query sequence (both stands) against a protein sequence database.
tblastn—compares a protein query sequence against a nucleotide sequence database dynamically translated in all six reading frames (both strands).
tblastx—compares the six-frame translations of a nucleotide query sequence against the six-frame translations of a nucleotide sequence database.
BLAST uses the following search parameters:
HISTOGRAM—Display a histogram of scores for each search; default is yes. (See parameter H in the BLAST Manual).
DESCRIPTIONS—Restricts the number of short descriptions of matching sequences reported to the number specified; default limit is 100 descriptions. (See parameter V in the manual page).
EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).
CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.
ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).
MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.
STRAND—Restrict a TBLASTN search to just the top or bottom strand of the database sequences; or restrict a BLASTN, BLASTX or TBLASTX search to just reading frames on the top or bottom strand of the query sequence.
FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see the website of the U.S. National Institutes of Health National Center for Biotechnology Information). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided the U.S. National Institutes of Health National Center for Biotechnology Information.

In some aspects of the present invention, no gap penalties are used when determining sequence identity.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Cloning and Expression

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

For recombinant production, host cells can be genetically engineered to incorporate expression systems or polynucleotides of the invention. Introduction of a polynucleotide into the host cell can be effected by methods described in many standard laboratory manuals, such as Davis et al and Sambrook et al, such as calcium phosphate transfection, DEAE-dextran mediated transfection, transfection, microinjection, cationic lipid-mediated transfection, electroporation, transduction, scrape loading, ballistic introduction and infection. It will be appreciated that such methods can be employed in vitro or in vivo as drug delivery systems.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

Polypeptides and Polynucleotides for Notch Signalling Transduction

The Notch signalling pathway directs binary cell fate decisions in the embryo. Notch was first described in Drosophila as a transmembrane protein that functions as a receptor for two different ligands, Delta and Serrate. Vertebrates express multiple Notch receptors and ligands (discussed below). At least four Notch receptors (Notch-1, Notch-2, Notch-3 and Notch-4) have been identified to date in human cells (see for example GenBank Accession Nos. AF308602, AF308601 and U95299—Homo sapiens).

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs in the Golgi apparatus and is mediated by a furin-like convertase.

Notch receptors are inserted into the membrane as heterodimeric molecules consisting of an extracellular domain containing up to 36 epidermal growth factor (EGF)-like repeats [Notch 1/2=36, Notch 3=34 and Notch 4=29], 3 Cysteine Rich Repeats (Lin-Notch (L/N) repeats) and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and is involved in binding to a transcription factor, known as Suppressor of Hairless [Su(H)] in Drosophila and CBF1 in vertebrates (Tamura). The Notch ligands also display multiple EGF-like repeats in their extracellular domains together with a cysteine-rich DSL (Delta-Serrate Lag2) domain that is characteristic of all Notch ligands (Artavanis-Tsakonas).

The Notch receptor is activated by binding of extracellular ligands, such as Delta, Serrate and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter). It is thought that the proteolytic cleavage step that releases the cdc10/ankyrin repeats for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

S3 processing occurs only in response to binding of Notch ligands Delta or Serrate/Jagged. The post-translational modification of the nascent Notch receptor in the Golgi (Munro; Ju) appears, at least in part, to control which of the two types of ligand is expressed on a cell surface. The Notch receptor is modified on its extracellular domain by Fringe, a glycosyl transferase enzyme that binds to the Lin/Notch motif. Fringe modifies Notch by adding O-linked fucose groups to the EGF-like repeats (Moloney; Bruckner). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin; Hicks). Although Drosophila has a single Fringe gene, vertebrates are known to express multiple genes (Radical, Manic and Lunatic Fringes) (Irvine).

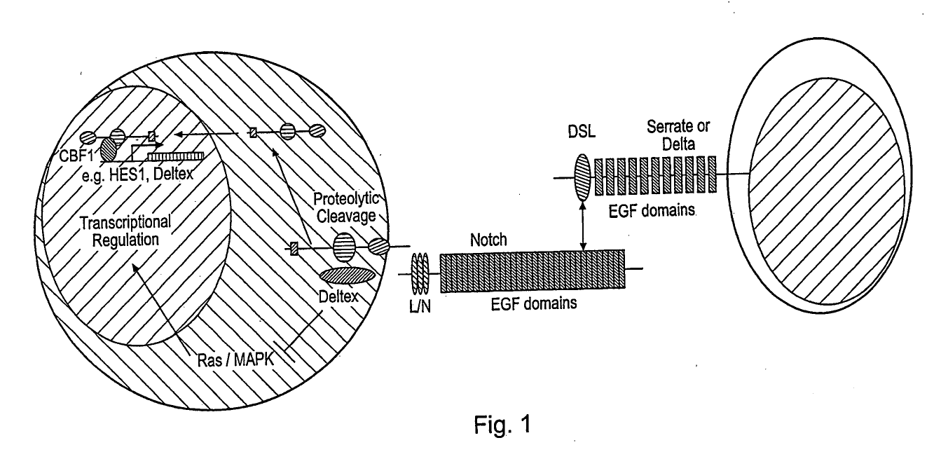

Signal transduction from the Notch receptor can occur via two different pathways (FIG. 1). The better defined pathway involves proteolytic cleavage of the intracellular domain of Notch (Notch IC) that translocates to the nucleus and forms a transcriptional activator complex with the CSL family protein CBF1 (suppressor of Hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltex. Unlike CBF1, Deltex does not move to the nucleus following Notch activation but instead can interact with Grb2 and modulate the Ras-JNK signalling pathway.

Thus, signal transduction from the Notch receptor can occur via two different pathways both of which are illustrated in FIG. 1. Target genes of the Notch signalling pathway include Deltex, genes of the Hes family (Hes-1 in particular), Enhancer of Split [E(spl)] complex genes, IL-10, CD-23, CD-4 and Dll-1.

Deltex, an intracellular docking protein, replaces Su(H) as it leaves its site of interaction with the intracellular tail of Notch. Deltex is a cytoplasmic protein containing a zinc-finger (Artavanis-Tsakonas; Osborne). It interacts with the ankyrin repeats of the Notch intracellular domain. Studies indicate that Deltex promotes Notch pathway activation by interacting with Grb2 and modulating the Ras-JNK signalling pathway (Matsuno). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayashi) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464.

The E(spl) gene complex [E(spl)-C] (Leimeister) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

Interleukin-10 (IL-10) was first characterised in the mouse as a factor produced by Th2 cells which was able to suppress cytokine production by Th1 cells. It was then shown that IL-10 was produced by many other cell types including macrophages, keratinocytes, B cells, Th0 and Th1 cells. It shows extensive homology with the Epstein-Barr bcrf1 gene which is now designated viral IL-10. Although a few immunostimulatory effects have been reported, it is mainly considered as an immunosuppressive cytokine. Inhibition of T cell responses by IL-10 is mainly mediated through a reduction of accessory functions of antigen presenting cells. IL-10 has notably been reported to suppress the production of numerous pro-inflammatory cytokines by macrophages and to inhibit co-stimulatory molecules and MHC class II expression. IL-10 also exerts anti-inflammatory effects on other myeloid cells such as neutrophils and eosinophils. On B cells, IL-10 influences isotype switching and proliferation. More recently, IL-10 was reported to play a role in the induction of regulatory T cells and as a possible mediator of their suppressive effect. Although it is not clear whether it is a direct downstream target of the Notch signalling pathway, its expression has been found to be strongly up-regulated coincident with Notch activation. The mRNA sequence of IL-10 may be found in GenBank ref. No. G11041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. The sequence for CD-23 may be found in GenBank ref. No. GI1783344.

Dlx-1 (distalless-1) (McGuiness) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

Other genes involved in the Notch signaling pathway, such as Numb, Mastermind and Dsh, and all genes the expression of which is modulated by Notch activation, are included in the scope of this invention.

Polypeptides and Polynucleotides for Notch Signalling Activation

Examples of mammalian Notch ligands identified to date include the Delta family, for example Delta-1 (Genbank Accession No. AF003522—Homo sapiens), Delta-3 (Genbank Accession No. AF084576—Rattus norvegicus) and Delta-like 3 (Mus musculus), the Serrate family, for example Serrate-1 and Serrate-2 (WO97/01571, WO096/27610 and WO92/19734), Jagged-1 and Jagged-2 (Genbank Accession No. AF029778—Homo sapiens), and LAG-2. Homology between family members is extensive.

Further homologues of known mammalian Notch ligands may be identified using standard techniques. By a “homologue” it is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand over a sequnce of at least 10, preferably at least 20, preferably at least 50, suitably at least 100 amino acids, or over the entire length of the Notch ligand. Techniques and software for calculating sequence homology between two or more amino acid or nucleic acid sequences are well known in the art (see for example databases maintained by the U.S. National Institutes of Health National Center for Biotechnology Information and Ausubel et al., Current Protocols in Molecular Biology (1995), John Wiley & Sons, Inc.)

Notch ligands identified to date have a diagnostic DSL domain (D. Delta, S. Serrate, L. Lag2) comprising 20 to 22 amino acids at the amino terminus of the protein and up to 14 or more EGF-like repeats on the extracellular surface. It is therefore preferred that homologues of Notch ligands also comprise a DSL domain at the N-terminus and up to 14 or more EGF-like repeats on the extracellular surface.

In addition, suitable homologues will be capable of binding to a Notch receptor. Binding may be assessed by a variety of techniques known in the art including in vitro binding assays.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Polypeptide substances may be purified from mammalian cells, obtained by recombinant expression in suitable host cells or obtained commercially. Alternatively, nucleic acid constructs encoding the polypeptides may be used. As a further example, overexpression of Notch or Notch ligand, such as Delta or Serrate, may be brought about by introduction of a nucleic acid construct capable of activating the endogenous gene, such as the Serrate or Delta gene. In particular, gene activation can be achieved by the use of homologous recombination to insert a heterologous promoter in place of the natural promoter, such as the Serrate or Delta promoter, in the genome of the target cell.

The activating molecule of the present invention may, in an alternative embodiment, be capable of modifying Notch-protein expression or presentation on the cell membrane or signalling pathways. Agents that enhance the presentation of a fully functional Notch-protein on the target cell surface include matrix metalloproteinases such as the product of the Kuzbanian gene of Drosophila (Dkuz) and other ADAMALYSIN gene family members.

Notch Ligand Domains

As discussed above, Notch ligands typically comprise a number of distinctive domains. Some predicted/potential domain locations for various naturally occurring human Notch ligands (based on amino acid numbering in the precursor proteins) are shown below:

Human Delta 1

| Component | Amino acids | Proposed function/domain |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| Component | Amino acids | Proposed function/domain |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| Component | Amino acids | Proposed function/domain |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| Component | Amino acids | Proposed function/domain |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND FACTOR C |

Human Jagged 2

| Component | Amino acids | Proposed function/domain |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND FACTOR C |

DSL Domain

A typical DSL domain may include most or all of the following consensus amino acid sequence (SEQ ID NO:22):

Cys Xaa Xaa Xaa Xaa Xaa Xaa Xaa Xaa Cys Xaa Xaa Xaa Cys Xaa Xaa
Xaa Xaa Xaa Xaa Xaa Xaa Xaa Xaa Xaa Cys Xaa Xaa Xaa Xaa Xaa Xaa
Xaa Cys Xaa Xaa Xaa Xaa Xaa Xaa Xaa Xaa Cys

Preferably the DSL domain may include most or all of the following consensus amino acid sequence (SEQ ID NO:23):

Cys Xaa Xaa Xaa ARO ARO Xaa Xaa Xaa Cys Xaa Xaa Xaa Cys BAS NOP
BAS ACM ACM Xaa ARO NOP ARO Xaa Xaa Cys Xaa Xaa Xaa NOP Xaa Xaa
Xaa Cys Xaa Xaa NOP ARO Xaa NOP Xaa Xaa Cys

wherein:
ARO is an aromatic amino acid residue, such as tyrosine, phenylalanine, tryptophan or histidine;
NOP is a non-polar amino acid residue such as glycine, alanine, proline, leucine, isoleucine or valine;
BAS is a basic amino acid residue such as arginine or lysine; and
ACM is an acid or amide amino acid residue such as aspartic acid, glutamic acid, asparagine or glutamine.

Preferably the DSL domain may include most or all of the following consensus amino acid sequence (SEQ ID NO:24):

Cys Xaa Xaa Xaa Tyr Tyr Xaa Xaa Xaa Cys Xaa Xaa Xaa Cys Arg Pro
Arg Asx Asp Xaa Phe Gly His Xaa Xaa Cys Xaa Xaa Xaa Gly Xaa Xaa
Xaa Cys Xaa Xaa Gly Trp Xaa Gly Xaa Xaa Cys

(wherein Xaa may be any amino acid and Asx is either aspartic acid or asparagine).

An alignment of DSL domains from Notch ligands from various sources is shown in the Figures.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

It will be appreciated that the term “DSL domain” as used herein includes sequence variants, fragments, derivatives and mimetics having activity corresponding to naturally occurring domains.

Suitably, for example, a DSL domain for use in the present invention may have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

EGF-like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors IX and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE a typical EGF domain may include six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of a typical EGF-like domain (SEQ ID NO:25):

```
                              +-------------------+ +-------------------------+
                              | | | |
x(4)-C-x(0,48)-C-x(3,12)-C-x(1,70)-C-x(1,6)-C-x(2)-G-a-x(0,21)-G-x(2)-C-x
          | | ************************************
          +-------------------+
```

wherein:
‘C’: conserved cysteine involved in a disulfide bond.
‘G’: often conserved glycine
‘a’: often conserved aromatic amino acid
‘*’: position of both patterns.
‘x’: any residue

The region between the 5th and 6th cysteine contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

It will be appreciated that the term “EGF domain” as used herein includes sequence variants, fragments, derivatives and mimetics having activity corresponding to naturally occurring domains.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

As a practical matter, whether any particular amino acid sequence is at least X% identical to another sequence can be determined conventionally using known computer programs. For example, the best overall match between a query sequence and a subject sequence, also referred to as a global sequence alignment, can be determined using a program such as the FASTDB computer program based on the algorithm of Brutlag et al. (Comp. App. Biosci. (1990) 6:237-245). In a sequence alignment the query and subject sequences are either both nucleotide sequences or both amino acid sequences. The result of the global sequence alignment is given as percent identity.

The term “Notch ligand N-terminal domain” means the part of a Notch ligand sequence from the N-terminus to the start of the DSL domain. It will be appreciated that this term includes sequence variants, fragments, derivatives and mimetics having activity corresponding to naturally occurring domains.

Suitably, for example, a Notch ligand N-terminal domain for use in the present invention may have at least 30%, preferably at least 50%, preferably at least 60%, preferably at least 70%, preferably at least 80%, preferably at least 90%, preferably at least 95% amino acid sequence identity to a Notch ligand N-terminal domain of human Jagged 1.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Jagged 2.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 1.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 3.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 4.

The term “heterologous amino acid sequence” or “heterologous nucleotide sequence“as used herein means a sequence which is not found in the native sequence (e.g. in the case of a Notch ligand sequence is not found in the native Notch ligand sequence) or its coding sequence. Typically, for example, such a sequence may be an IgFc domain or a tag such as a V5His tag.

Notch signalling can be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. In particular, an increase in levels of Deltex, Hes-1 and/or IL-10 mRNA may, for instance, indicate induced anergy while an increase in levels of Dll-1 or IFN-γ mRNA, or in the levels of mRNA encoding cytokines such as IL-2, IL-5 and IL-13, may indicate improved responsiveness.

Various nucleic acid assays are known. Any convention technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

In particular, gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefore easily identifiable.

In general, reporter constructs useful for detecting Notch signalling by expression of a reporter gene may be constructed according to the general teaching of Sambrook et al. (1989). Typically, constructs according to the invention comprise a promoter by the gene of interest, and a coding sequence encoding the desired reporter constructs, for example of GFP or luciferase. Vectors encoding GFP and luciferase are known in the art and available commercially.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefore assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al. (1989).

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In vivo Expression Technology (IVET) (Camilli). IVET identifies genes up-regulated during say treatment or disease when compared to laboratory culture.

The advantage of using a protein assay is that Notch activation can be directly measured. Assay techniques that can be used to determine levels of a polypeptide are well known to those skilled in the art. Such assay methods include radioimmunoassays, competitive-binding assays, Western Blot analysis, antibody sandwich assays, antibody detection, FACS and ELISA assays.

As described above the modulator of Notch signalling may also be an immune cell which has been treated to modulate expression or interaction of Notch, a Notch ligand or the Notch signalling pathway. Such cells may readily be prepared, for example, as described in WO 00/36089 in the name of Lorantis Ltd, the text of which is herein incorporated by reference.

Chemical Cross-Linking

It will be appreciated that multimers may be prepared for example by chemical cross-linking or generic engineering techniques.

Chemically coupled (cross-linked) sequences can be prepared from individual protein or polypeptide sequences and coupled using known chemical coupling techniques. A conjugate can for example be assembled using conventional solution- or solid-phase peptide synthesis methods, affording a fully protected precursor with only the terminal amino group in deprotected reactive form. This function can then be reacted directly with a protein for Notch signalling modulation or a suitable reactive derivative thereof. Alternatively, this amino group may be converted into a different functional group suitable for reaction with a cargo moiety or a linker. Thus, e.g. reaction of the amino group with succinic anhydride will provide a selectively addressable carboxyl group, while further peptide chain extension with a cysteine derivative will result in a selectively addressable thiol group. Once a suitable selectively addressable functional group has been obtained in the delivery vector precursor, a protein for Notch signalling modulation or a derivative thereof may be attached through e.g. amide, ester, or disulphide bond formation. Cross-linking reagents which can be utilized are discussed, for example, in Means, G. E. and Feeney, R. E., Chemical Modification of Proteins, Holden-Day, 1974, pp. 39-43.

As discussed above the polymer and proteins or polypeptides for Notch signalling modulation may be linked directly or indirectly suitably via a linker moiety. Direct linkage may occur through any convenient functional group on the protein for Notch signalling modulation such as a thiol, hydroxy, carboxy or amino group. Indirect linkage which is may sometimes be preferable, will occur through a linking moiety. Suitable linking moieties include bi- and multi-functional alkyl, aryl, aralkyl or peptidic moieties, alkyl, aryl or aralkyl aldehydes acids esters and anyhdrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or sulphonic halides and the like. The functional groups on the linker moiety used to form covalent bonds between linker and proteins for Notch signalling modulation may be two or more of, e.g., amino, hydrazino, hydroxyl, thiol, maleimido, carbonyl, and carboxyl groups, etc. The linker moiety may include a short sequence of e.g. from 1 to 4 amino acid residues that optionally includes a cysteine residue through which the linker moiety bonds to the target protein or polypeptide.

Therapeutic Uses

A. Immunoloical uses of the Present Invention

In a preferred embodiment, the constructs of the present invention may be used to modify immune responses in the immune system of a mammal, such as a human. Preferably such modulation of the immune system is effected by control of immune cell, preferably T-cell, preferably peripheral T-cell, activity.

A detailed description of the Notch signalling pathway and conditions affected by it may be found in our WO98/20142, WO00/36089 and PCT/GB00/04391.

Diseased or infectious states that may be described as being mediated by T cells include, but are not limited to, any one or more of asthma, allergy, graft rejection, autoimmunity, tumour induced aberrations to the T cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T cells include multiple schlerosis, rheumatoid arthritis and diabetes. The present invention may also be used in organ transplantation or bone marrow transplantation.

As indicated above, the present invention is useful in treating immune disorders such as autoimmune diseases or graft rejection such as allograft rejection.

Autoimmune Disease

Examples of disorders that may be treated include a group commonly called autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail: Organ-specific autoimmune diseases include multiple sclerosis, insulin dependent diabetes mellitus, several forms of anemia (aplastic, hemolytic), autoimmune hepatitis, thyroiditis, insulitis, iridocyclitis, scleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis.

A more extensive list of disorders includes: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

Transplant Rejection

The present invention may be used, for example, for the treatment of organ transplants (e.g. kidney, heart, lung, liver or pancreas transplants), tissue transplants (e.g. skin grafts) or cell transplants (e.g. bone marrow transplants or blood transfusions).

A brief overview of the most common types of organ and tissue transplants is set out below.

i) Kidney Transplants

Kidneys are the most commonly transplanted organs. Kidneys can be donated by both cadavers and living donors and kidney transplants can be used to treat numerous clinical indications (including diabetes, various types of nephritis and kidney failure). Surgical procedure for kidney transplantation is relatively simple. However, matching blood types and histocompatibility groups is desirable to avoid graft rejection. It is indeed important that a graft is accepted as many patients can become “sensitised” after rejecting a first transplant. Sensitisation results in the formation of antibodies and the activation of cellular mechanisms directed against kidney antigens. Thus, any subsequent graft containing antigens in common with the first is likely to be rejected. As a result, many kidney transplant patients must remain on some form of immunosuppressive treatment for the rest of their lives, giving rise to complications such as infection and metabolic bone disease.

ii) Heart Transplantation

Heart transplantation is a very complex and high-risk procedure. Donor hearts must be maintained in such a manner that they will begin beating when they are placed in the recipient and can therefore only be kept viable for a limited period under very specific conditions. They can also only be taken from brain-dead donors. Heart transplants can be used to treat various types of heart disease and/or damage. HLA matching is obviously desirable but often impossible because of the limited supply of hearts and the urgency of the procedure.

iii) Lung Transplantation

Lung transplantation is used (either by itself or in combination with heart transplantation) to treat diseases such as cystic fibrosis and acute damage to the lungs (e.g. caused by smoke inhalation). Lungs for use in transplants are normally recovered from brain-dead donors.

iv) Pancreas Transplantation

Pancreas transplantation is mainly used to treat diabetes mellitus, a disease caused by malfunction of insulin-producing islet cells in the pancreas. Organs for transplantation can only be recovered from cadavers although it should be noted that transplantation of the complete pancreas is not necessary to restore the function needed to produce insulin in a controlled fashion. Indeed, transplantation of the islet cells alone could be sufficient. Because kidney failure is a frequent complication of advanced diabetes, kidney and pancreas transplants are often carried out simultaneously.

v) Skin Grafting

Most skin transplants are done with autologous tissue. However, in cases of severe burning (for example), grafts of foreign tissue may be required (although it should be noted that these grafts are generally used as biological dressings as the graft will not grow on the host and will have to be replaced at regular intervals). In cases of true allogenic skin grafting, rejection may be prevented by the use of immunosuppressive therapy. However, this leads to an increased risk of infection and is therefore a major drawback in burn victims.

vi) Liver Transplantation

Liver transplants are used to treat organ damage caused by viral diseases such as hepititis, or by exposure to harmful chemicals (e.g. by chronic alcoholism). Liver transplants are also used to treat congenital abnormalities. The liver is a large and complicated organ meaning that transplantation initially posed a technical problem. However, most transplants (65%) now survive for more than a year and it has been found that a liver from a single donor may be split and given to two recipients. Although there is a relatively low rate of graft rejection by liver transplant patients, leukocytes within the donor organ together with anti-blood group antibodies can mediate antibody-dependent hemolysis of recipient red blood cells if there is a mismatch of blood groups. In addition, manifestations of GVHD have occurred in liver transplants even when donor and recipient are blood-group compatible.

Vaccines and Cancer Vaccines

The constructs of the present invention may also be used in vaccine compositions such as cancer and pathogen vaccines.

Vaccine Compositions

Conjugates according to the present invention which inhibit Notch signalling may be employed in vaccine compositions (such as pathogen or cancer vaccines) to protect or treat a mammal susceptible to, or suffering from disease, by means of administering said vaccine via a mucosal route, such as the oral/bucal/intestinal/vaginal/rectal or nasal route. Such administration may for example be in a droplet, spray, or dry powdered form. Nebulised or aerosolised vaccine formulations may also be used where appropriate.

Enteric formulations such as gastro resistant capsules and granules for oral administration, suppositories for rectal or vaginal administration may also be used. The present invention may also be used to enhance the immunogenicity of antigens applied to the skin, for example by intradermal, transdermal or transcutaneous delivery. In addition, the adjuvants of the present invention may be parentally delivered, for example by intramuscular or subcutaneous administration.

Depending on the route of administration, a variety of administration devices may be used. For example, for intranasal administration a spray device such as the commercially available Accuspray (Becton Dickinson) may be used.

Preferred spray devices for intranasal use are devices for which the performance of the device is not dependent upon the pressure applied by the user. These devices are known as pressure threshold devices. Liquid is released from the nozzle only when a threshold pressure is attained. These devices make it easier to achieve a spray with a regular droplet size. Pressure threshold devices suitable for use with the present invention are known in the art and are described for example in WO 91/13281 and EP 311 863 B. Such devices are commercially available from Pfeiffer GmbH.

For certain vaccine formulations, other vaccine components may be included in the formulation. For example the adjuvant formulations of the present invention may also comprise a bile acid or derivative of cholic acid. Suitably the derivative of cholic acid is a salt thereof, for example a sodium salt thereof. Examples of bile acids include cholic acid itself, deoxycholic acid, chenodeoxy colic acid, lithocholic acid, taurodeoxycholate ursodeoxycholic acid, hyodeoxycholic acid and derivatives like glyco-, tauro-, amidopropyl-1-propanesulfonic- and amidopropyl-2-hydroxy-1-propanesulfonic-derivatives of the above bile acids, or N,N-bis(3DGluconoamidopropyl)deoxycholamide.

Suitably, an adjuvant formulation of the present invention may be in the form of an aqueous solution or a suspension of non-vesicular forms. Such formulations are convenient to manufacture, and also to sterilise (for example by terminal filtration through a 450 or 220 nm pore membrane).

Suitably, the route of administration may be via the skin, intramuscular or via a mucosal surface such as the nasal mucosa. When the admixture is administered via the nasal mucosa, the admixture may for example be administered as a spray. The methods to enhance an immune response may be either a priming or boosting dose of the vaccine.

The term “adjuvant” as used herein includes an agent having the ability to enhance the immune response of a vertebrate subject's immune system to an antigen or antigenic determinant.

The term “immune response” includes any response to an antigen or antigenic determinant by the immune system of a subject. Immune responses include for example humoral immune responses (e.g. production of antigen-specific antibodies) and cell-mediated immune responses (e.g. lymphocyte proliferation).

The term “cell-mediated immune response” includes the immunological defence provided by lymphocytes, such as the defence provided by T cell lymphocytes when they come into close proximity with their victim cells.

When “lymphocyte proliferation” is measured, the ability of lymphocytes to proliferate in response to specific antigen may be measured. Lymphocyte proliferation includes B cell, T-helper cell or CTL cell proliferation.

Compositions of the present invention may be used to formulate vaccines containing antigens derived from a wide variety of sources. For example, antigens may include human, bacterial, or viral nucleic acid, pathogen derived antigen or antigenic preparations, host-derived antigens, including GnRH and IgE peptides, recombinantly produced protein or peptides, and chimeric fusion proteins.

Preferably the vaccine formulations of the present invention contain an antigen or antigenic composition capable of eliciting an immune response against a human pathogen. The antigen or antigens may, for example, be peptides/proteins, polysaccharides and lipids and may be derived from pathogens such as viruses, bacteria and parasites/fungi as follows:

Viral Antigens

Viral antigens or antigenic determinants may be derived, for example, from:

Cytomegalovirus (especially Human, such as gB or derivatives thereof); Epstein Barr virus (such as gp350); flaviviruses (e.g. Yellow Fever Virus, Dengue Virus, Tick-borne encephalitis virus, Japanese Encephalitis Virus); hepatitis virus such as hepatitis B virus (for example Hepatitis B Surface antigen such as the PreS1, PreS2 and S antigens described in EP-A-414 374; EP-A-0304 578, and EP-A-198474), hepatitis A virus, hepatitis C virus and hepatitis E virus; HIV-1, (such as tat, nef, gp120 or gp160); human herpes viruses, such as gD or derivatives thereof or Immediate Early protein such as ICP27 from HSV1 or HSV2; human papilloma viruses (for example HPV6, 11, 16, 18); Influenza virus (whole live or inactivated virus, split influenza virus, grown in eggs or MDCK cells, or Vero cells or whole flu virosomes (as described by Gluck, Vaccine, 1992,10, 915-920) or purified or recombinant proteins thereof, such as NP, NA, HA, or M proteins); measles virus; mumps virus; parainfluenza virus; rabies virus; Respiratory Syncytial virus (such as F and G proteins); rotavirus (including live attenuated viruses); smallpox virus; Varicella Zoster Virus (such as gpI, II and IE63); and the HPV viruses responsible for cervical cancer (for example the early proteins E6 or E7 in fusion with a protein D carrier to form Protein D-E6 or E7 fusions from HPV 16, or combinations thereof; or combinations of E6 or E7 with L2 (see for example WO 96/26277).

Bacterial Antigens

Bacterial antigens or antigenic determinants may be derived, for example, from:

Bacillus spp., including B. anthracis (e.g. botulinum toxin); Bordetella spp, including B. pertussis (for example pertactin, pertussis toxin, filamenteous hemagglutinin, adenylate cyclase, fimbriae); Borrelia spp., including B. burgdorferi (e.g. OspA, OspC, DbpA, DbpB), B. garinii (e.g. OspA, OspC, DbpA, DbpB), B. afzelii (e.g. OspA, OspC, DbpA, DbpB), B. andersonii (e.g. OspA, OspC, DbpA, DbpB), B. hermsii; Campylobacter spp, including C. jejuni (for example toxins, adhesins and invasins) and C. coli;

Chlamydia spp., including C. trachomatis (e.g. MOMP, heparin-binding proteins), C. pneumonie (e.g. MOMP, heparin-binding proteins), C. psittaci; Clostridium spp., including C. tetani (such as tetanus toxin), C. botulinum (for example botulinum toxin), C. difficile (e.g. clostridium toxins A or B); Corynebacterium spp., including C. diphtheriae (e.g. diphtheria toxin); Ehrlichia spp., including E. equi and the agent of the Human Granulocytic Ehrlichiosis; Rickettsia spp, including R.rickettsii;

Enterococcus spp., including E. faecalis, E. faecium; Escherichia spp, including enterotoxic E. coli (for example colonization factors, heat-labile toxin or derivatives thereof, or heat-stable toxin), enterohemorragic E. coli, enteropathogenic E. coli (for example shiga toxin-like toxin); Haemophilus spp., including H. influenzae type B (e.g. PRP), non-typable H. influenzae, for example OMP26, high molecular weight adhesins, P5, P6, protein D and lipoprotein D, and fimbrin and fimbrin derived peptides (see for example U.S. Pat. No. 5,843,464); Helicobacter spp, including H. pylori (for example urease, catalase, vacuolating toxin); Pseudomonas spp, including P. aeruginosa;

Legionella spp, including L. pneumophila; Leptospira spp., including L. interrogans;

Listeria spp., including L. monocytogenes; Moraxella spp, including M catarrhalis, also known as Branhamella catarrhalis (for example high and low molecular weight adhesins and invasins); Morexella Catarrhalis (including outer membrane vesicles thereof, and OMP106 (see for example WO97/41731)); Mycobacterium spp., including M. tuberculosis (for example ESAT6, Antigen 85A, -B or -C), M. bovis, M. leprae, M. avium, M. paratuberculosis, M. smegmatis; Neisseria spp, including N. gonorrhea and N. meningitidis (for example capsular polysaccharides and conjugates thereof, transferrin-binding proteins, lactoferrin binding proteins, PilC, adhesins); Neisseria mengitidis B (including outer membrane vesicles thereof, and NspA ( see for example WO 96/29412); Salmonella spp, including S. typhi, S. paratyphi, S. choleraesuis, S. enteritidis; Shigella spp, including S. sonnei, S. dysenteriae, S. flexnerii; Staphylococcus spp., including S. aureus, S. epidermidis; Streptococcus spp, including S. pneumonie (e.g. capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (see for example WO 90/06951; WO 99/03884); Treponema spp., including T. pallidum (e.g. the outer membrane proteins), T. denticola, T. hyodysenteriae; Vibrio spp, including V. cholera (for example cholera toxin); and Yersinia spp, including Y. enterocolitica (for example a Yop protein), Y. pestis, Y. pseudotuberculosis.

Parasite/Fungal Antigens

Parasitic/fungal antigens or antigenic determinants may be derived, for example, from:
Babesia spp., including B. microti; Candida spp., including C. albicans;
Cryptococcus spp., including C. neoformans; Entamoeba spp., including E. histolytica;
Giardia spp., including; G. lamblia; Leshmania spp., including L. major;
Plasmodium. faciparum (MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.);
Pneumocystis spp., including P. carinii; Schisostoma spp., including S. mansoni;
Trichomonas spp., including T. vaginalis; Toxoplasma spp., including T. gondii (for example SAG2, SAG3, Tg34); Trypanosoma spp., including T. cruzi.

Approved/licensed vaccines include, for example anthrax vaccines such as Biothrax (BioPort Corp); tuberculosis (BCG) vaccines such as TICE BCG (Organon Teknika Corp) and Mycobax (Aventis Pasteur, Ltd); diphtheria & tetanus toxoid and acellular pertussis (DTP) vaccines such as Tripedia (Aventis Pasteur, Inc), Infanrix (GlaxoSmithKline), and DAPTACEL (Aventis Pasteur, Ltd); Haemophilus b conjugate vaccines (e.g. diphtheria CRM197 protein conjugates such as HibTITER from Lederle Lab Div, American Cyanamid Co; meningococcal protein conjugates such as PedvaxHIB from Merck & Co, Inc; and tetanus toxoid conjugates such as ActHIB from Aventis Pasteur, SA); Hepatitis A vaccines such as Havrix (GlaxoSmithKline) and VAQTA (Merck & Co, Inc); combined Hepatitis A and Hepatitis B (recombinant) vaccines such as Twinrix (GlaxoSmithKline); recombinant Hepatitis B vaccines such as Recombivax HB (Merck & Co, Inc) and Engerix-B (GlaxoSmithKline); influenza virus vaccines such as Fluvirin (Evans Vaccine), FluShield (Wyeth Laboratories, Inc) and Fluzone (Aventis Pasteur, Inc); Japanese Encephalitis virus vaccine such as JE-Vax (Research Foundation for Microbial Diseases of Osaka University); Measles virus vaccines such as Attenuvax (Merck & Co, Inc); measles and mumps virus vaccines such as M-M-Vax (Merck & Co, Inc); measles, mumps, and rubella virus vaccines such as M-M-R II (Merck & Co, Inc); meningococcal polysaccharide vaccines (Groups A, C, Y and W-135 combined) such as Menomune-A/C/Y/W-135 (Aventis Pasteur, Inc); mumps virus vaccines such as Mumpsvax (Merck & Co, Inc); pneumococcal vaccines such as Pneumovax (Merck & Co, Inc) and Pnu-Imune (Lederle Lab Div, American Cyanamid Co); Pneumococcal 7-valent conjugate vaccines (e.g. diphtheria CRM197 Protein conjugates such as Prevnar from Lederle Lab Div, American Cyanamid Co); poliovirus vaccines such as Poliovax (Aventis Pasteur, Ltd); poliovirus vaccines such as IPOL (Aventis Pasteur, S A); rabies vaccines such as Imovax (Aventis Pasteur, S A) and RabAvert (Chiron Behring GmbH & Co); rubella virus vaccines such as Meruvax II (Merck & Co, Inc); Typhoid Vi polysaccharide vaccines such as TYPHIM Vi (Aventis Pasteur, S A); Varicella virus vaccines such as Varivax (Merck & Co, Inc) and Yellow Fever vaccines such as YF-Vax (Aventis Pasteur, Inc).

Cancer/Tumour Antigens

The term “cancer antigen or antigenic determinant” or “tumour antigen or antigenic determinant” as used herein preferably means an antigen or antigenic determinant which is present on (or associated with) a cancer cell and not typically on normal cells, or an antigen or antigenic determinant which is present on cancer cells in greater amounts than on normal (non-cancer) cells, or an antigen or antigenic determinant which is present on cancer cells in a different form than that found on normal (non-cancer) cells.

Cancer antigens include, for example (but without limitation):
beta chain of human chorionic gonadotropin (hCG beta) antigen, carcinoembryonic antigen, EGFRvIII antigen, Globo H antigen, GM2 antigen, GP100 antigen, HER2/neu antigen, KSA antigen, Le (y) antigen, MUC1 antigen, MAGE 1 antigen, MAGE 2 antigen, MUC2 antigen, MUC3 antigen, MUC4 antigen, MUC5AC antigen, MUC5B antigen, MUC7 antigen, PSA antigen, PSCA antigen, PSMA antigen,

Thompson-Friedenreich antigen (TF), Tn antigen, sTn antigen, TRP 1 antigen, TRP 2 antigen, tumor-specific immunoglobulin variable region and tyrosinase antigen.

It will be appreciated that in accordance with this aspect of the present invention antigens and antigenic determinants may be used in many different forms. For example, antigens or antigenic determinants may be present as isolated proteins or peptides (for example in so-called “subunit vaccines”) or, for example, as cell-associated or virus-associated antigens or antigenic determinants (for example in either live or killed pathogen strains). Live pathogens will preferably be attenuated in known manner. Alternatively, antigens or antigenic determinants may be generated in situ in the subject by use of a polynucleotide coding for an antigen or antigenic determinant (as in so-called “DNA vaccination”, although it will be appreciated that the polynucleotides which may be used with this approach are not limited to DNA, and may also include RNA and modified polynucleotides as discussed above).

B. Non-Immunological uses of the Present Invention

Cell Fate/Cancer Indications

It will be appreciated however that the constructs of the present invention, as modulators of Notch sigalling, may also be used for altering the fate of a cell, tissue or organ type by altering Notch pathway function in a cell by a partially or fully non-immunological mode of action (e.g. by modifying general cell fate, differentiation or proliferation), as described, for example in WO 92/07474, WO 96/27610, WO 97/01571, U.S. Pat. No. 5,648,464, U.S. Pat. No. 5,849,869 and U.S. Pat. No. 6,004,924 (Yale University/Imperial Cancer Technology), the texts of which are herein incorporated by reference.

Thus, the conjugates of the present invention are also useful in methods for altering the fate of any cell, tissue or organ type by altering Notch pathway function in the cell. Thus, for example, the present constructs also have application in the treatment of malignant and pre-neoplastic disorders for example by an antiproliferative, rather than immunological mechanism. For example, in the cancer field the conjugates of the present invention are especially useful in relation to adenocarcinomas such as: small cell lung cancer, and cancer of the kidney, uterus, prostrate, bladder, ovary, colon and breast. For example, malignancies which may be treatable according to the present invention include acute and chronic leukemias, lymphomas, myelomas, sarcomas such as Fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimyosarcoma, rhabdomyosarcoma, colon carcinoma, ovarian cancer, prostate cancer, pancreatic cancer, breasy cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

The present invention may also have application in the treatment of nervous system disorders. Nervous system disorders which may be treated according to the present invention include neurological lesions including traumatic lesions resulting from physical injuries; ischaemic lesions; malignant lesions; infectious lesions such as those caused by HIV, herpes zoster or herpes simplex virus, Lyme disease, tuberculosis or syphilis; degenerative lesions and diseases and demyelinated lesions.

The present invention may be used to treat, for example, diabetes (including diabetic neuropathy, Bell's palsy), systemic lupus erythematosus, sarcoidosis, multiple sclerosis, human immunodeficiency virus-associated myelopathy, transverse myelopathy or various etiologies, progressive multifocal leukoencephalopathy, central pontine myelinolysis, Parkinson's disease, Alzheimer's disease, Huntington's chorea, amyotrophic lateral sclerosis, cerebral infarction or ischemia, spinal cord infarction or ischemia, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

The present invention may further be useful in the promotion of tissue regeneration and repair, for example by modification of differentiation processes. The present invention, therefore, may also be used to treat diseases associated with defective tissue repair and regeneration such as, for example, cirrhosis of the liver, hypertrophic scar formation and psoriasis. The invention may also be useful in the treatment of neutropenia or anemia and in techniques of organ regeneration and tissue engineering and stem cell treatments.

Pharmaceutical Compositions

Preferably the active agents of the present invention are administered in the form of pharmaceutical compositions. The pharmaceutical compositions may be for human or animal usage in human and veterinary medicine and in addition to one or more active agents will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro ed. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s). Preservatives, stabilizers, dyes and even flavoring agents may also be provided in such a pharmaceutical composition. Examples of preservatives include sodium benzoate, sorbic acid and esters of p-hydroxybenzoic acid. Antioxidants and suspending agents may be also used.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

In one embodiment the therapeutic agents used in the present invention may be administered directly to patients in vivo. Alternatively or in addition, the agents may be administered to cells (such as T cells and/or APCs or stem or tissue cells) in an ex vivo manner. For example, leukocytes such as T cells or APCs may be obtained from a patient or donor in known manner, treated/incubated ex vivo in the manner of the present invention, and then administered to a patient.

In general, a therapeutically effective daily dose may for example range from 0.01 to 500 mg/kg, for example 0.01 to 50 mg/kg body weight of the subject to be treated, for example 0.1 to 20 mg/kg. The conjugate of the present invention may also be administered by intravenous infusion, at a dose which is likely to range from for example 0.001-10 mg/kg/hr.

A skilled practitioner will be able to determine readily the optimum route of administration and dosage for any particular patient depending on, for example, the age, weight and condition of the patient. Preferably the pharmaceutical compositions are in unit dosage form.

The agents of the present invention can be administered by any suitable means including, but not limited to, for example, oral, rectal, nasal, topical (including intradermal, transdermal, aerosol, buccal and sublingual), vaginal and parenteral (including subcutaneous, intramuscular, intravenous and intradermal) routes of administration.

Suitably the active agents are administered in combination with a pharmaceutically acceptable carrier or diluent as described under the heading “Pharmaceutical compositions” above. The pharmaceutically acceptable carrier or diluent may be, for example, sterile isotonic saline solutions, or other isotonic solutions such as phosphate-buffered saline. The conjugates of the present invention may suitably be admixed with any suitable binder(s), lubricant(s), suspending agent(s), coating agent(s), solubilising agent(s).

In one embodiment, it may be desired to formulate the compound in an orally active form. Thus, for some applications, active agents may be administered orally in the form of tablets containing excipients such as starch or lactose, or in capsules or ovules either alone or in admixture with excipients, or in the form of elixirs, solutions or suspensions containing flavouring or colouring agents. Doses such as tablets or capsules comprising the conjugates may be administered singly or two or more at a time, as appropriate. It is also possible to administer the conjugates in sustained release formulations.

Alternatively or in addition, active agents may be administered by inhalation, intranasally or in the form of aerosol, or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, for example at a concentration of between 1 and 10% by weight, into an ointment consisting of a white wax or white soft paraffin base together with such stabilisers and preservatives as may be required.

Active agents such as polynucleotides and proteins/polypeptides may also be administered by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include, but are not limited to, mucosal, nasal, oral, parenteral, gastrointestinal, topical, or sublingual routes. Active agents may also be adminstered by needleless systems, such as ballistic delivery on particles for delivery to the epidermis or dermis or other sites such as mucosal surfaces.

Active agents may also be injected parenterally, for example intracavernosally, intravenously, intramuscularly or subcutaneously

For parenteral administration, active agents may for example be used in the form of a sterile aqueous solution which may contain other substances, for example enough salts or monosaccharides to make the solution isotonic with blood.

For buccal or sublingual administration, agents may for example be administered in the form of tablets or lozenges which can be formulated in a conventional manner.

For oral, parenteral, buccal and sublingual administration to subjects (such as patients), the dosage level of active agents and their pharmaceutically acceptable salts and solvates may typically be from 10 to 500 mg (in single or divided doses). Thus, and by way of example, tablets or capsules may contain from 5 to 100 mg of active agent for administration singly, or two or more at a time, as appropriate. As indicated above, the physician will determine the actual dosage which will be most suitable for an individual patient and it will vary with the age, weight and response of the particular patient. It is to be noted that whilst the above-mentioned dosages are exemplary of the average case there can, of course, be individual instances where higher or lower dosage ranges are merited and such dose ranges are within the scope of this invention.

The routes of administration and dosages described are intended only as a guide since a skilled practitioner will be able to determine readily the optimum route of administration and dosage for any particular patient depending on, for example, the age, weight and condition of the patient.

The term treatment or therapy as used herein should be taken to encompass diagnostic and prophylatic applications.

The treatment of the present invention includes both human and veterinary applications.

The active agents of the present invention may also be administered with other active agents such as, for example, immunosuppressants, steroids or anticancer agents.

Antigens and Allergens

In one embodiment, the active agents of the present invention may be administered in simultaneous, separate or sequential combination with antigens or antigenic determinants (or polynucleotides coding therefor), to modify (increase or decrease) the immune response to such antigens or antigenic determinants.

An antigen suitable for use in the present invention may be any substance that can be recognised by the immune system, and is generally recognised by an antigen receptor.

Preferably the antigen used in the present invention is an immunogen. An allergic response occurs when the host is re-exposed to an antigen that it has encountered previously.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (TH1) is characterised by high IL-2 and IFNγ but low IL-4 production, whereas in humoral immunity (TH2) the pattern is low IL-2 and IFNγ but high IL-4, IL-5 and IL-13. Since the secretory pattern is modulated at the level of the secondary lymphoid organ or cells, then pharmacological manipulation of the specific TH cytokine pattern can influence the type and extent of the immune response generated.

The TH1-TH2 balance refers to the relative representation of the two different forms of helper T cells. The two forms have large scale and opposing effects on the immune system. If an immune response favours TH1 cells, then these cells will drive a cellular response, whereas TH2 cells will drive an antibody-dominated response. The type of antibodies responsible for some allergic reactions is induced by TH2 cells.

The antigen or allergen (or antigenic determinant thereof) used in the present invention may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component. In particular, it is preferred to use antigens known to be associated with auto-immune diseases such as myelin basic protein (associated with multiple sclerosis), collagen (associated with rheumatoid arthritis), and insulin (diabetes), or antigens associated with rejection of non-self tissue such as MHC antigens or antigenic determinants thereof. Where primed the APCs and/or T cells of the present invention are to be used in tissue transplantation procedures, antigens may be obtained from the tissue donor. Polynucleotides coding for antigens or antigenic determinants which may be expessed in a subject may also be used.

Various preferred features and embodiments of the present invention will now be described in more detail by way of non-limiting examples.

### EXAMPLES

### Example 1

### Delta 1 DSL Domain Plus EGF Repeats 1-2

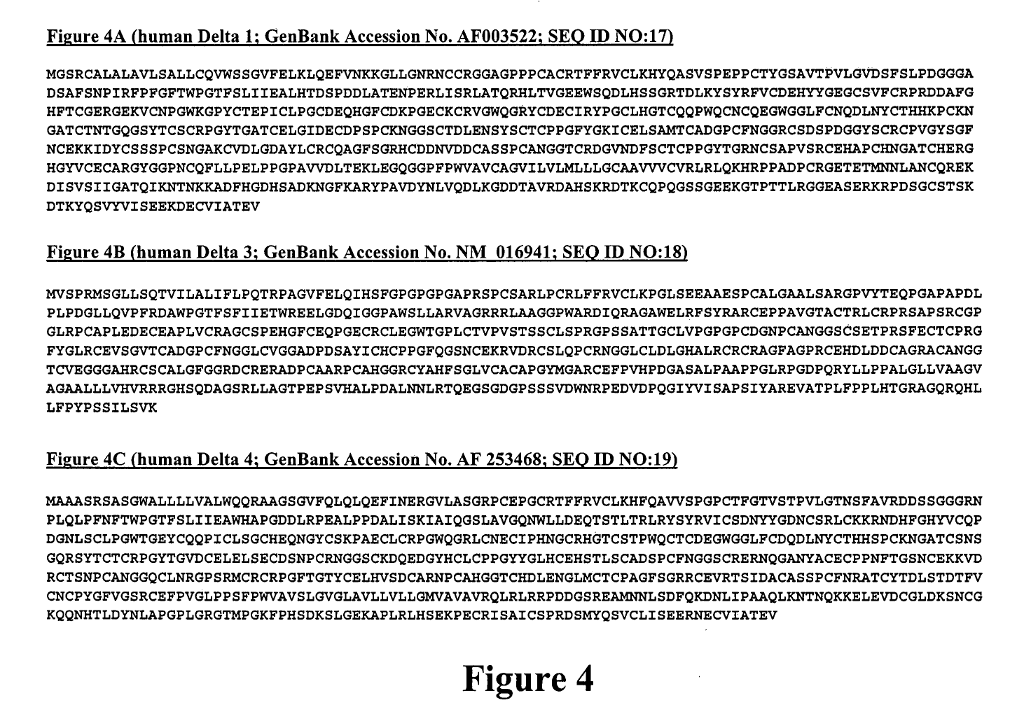

A human Delta 1 (DLL-1) deletion coding for the DSL domain and the first two only of the naturally occurring EGF repeats (i.e. omitting EGF repeats 3 to 8 inclusive) was generated by PCR from a DLL-1 extracellular (EC) domain/V5His clone (for the sequence of the human DLL-1 EC domain see FIG. 4 and, for example, Genbank Accession No. AF003522) using a primer pair as follows:

| | |
|---|---|
| DLac13: | |
| CACCAT GGGCAG TCGGTG CGCGCT GG | (SEQ ID NO:26) |
| and | |
| DLL1d3-8: | |
| GTAGTT CAGGTC CTGGTT GCAG | (SEQ ID NO:27) |

PCR conditions were:
1 cycle at 95° C./3 minutes;
18 cycles of(95° C./1 minute, 60° C./1 minute, 72° C./2 minutes); and
1 cycle at 72° C./2 minutes.

The DNA was then isolated from a 1% agarose gel in 1×U/V-Safe TAE (Tris/acetate/EDTA) buffer (MWG-Biotech, Ebersberg, Germany) and used as a template for PCR with the following primers:

FcDL.4:

(SEQ ID NO:28)

CACCAT GGGCAG TCGGTG CGCGCT GG
and
FcDLLd3-8:

(SEQ ID NO:29)

GGATAT GGGCCC TTGGTG GAAGCG TAGTTC AGGTCC TGGTTG
CAG

PCR conditions were:
1 cycle at 94° C./3 minutes;
18 cycles of(94° C./1 minute, 68° C./1 minute, 72° C./2 minutes); and
1 cycle at 72° C./10 minutes.

The fragment was ligated into pCRbluntII.TOPO (Invitrogen) and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with ApaI and HindIII then treated with shrimp alkaline phosphatase (Roche) and gel purified.

The DLL-1 deletions cloned in pCRbluntII were cut with HindIII (and EcoRV to aid later selection of the desired DNA product) followed by ApaI partial restriction. The sequences were then gel purified and ligated into the pCONγ vector which was cloned into TOP10 cells.

Plasmid DNA was generated using a Qiagen Minprep kit (QIAprep™) according to the manufacturer's instructions.

The resulting construct (pCON_{χ} hDLL1 EGF1-2) coded for the following DLL-1 amino acid sequence (SEQ D NO:30) fused to the IgG Fc domain encoded by the pCONγ vector.

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNY

(wherein the emboldened portion of the sequence which is single underlined is the DSL domain and the emboldened portions of the sequence which are double underlined are EGF repeats 1 and 2 respectively).

### Example 2

### Delta 1 DSL Domain Plus EGF repeats 1-3

A human Delta 1 (DLL-1) deletion coding for the DSL domain and the first three only of the naturally occuring EGF repeats (i.e. omitting EGF repeats 4 to 8 inclusive) was generated by PCR from a DLL-1 DSL plus EGF repeats 1-4 clone using a primer pair as follows:

DLac13:

(SEQ ID NO:31)

CACCATGGGCAGTCGGTGCGCGCTGG
and
FcDLLd4-8:

(SEQ ID NO:32)

GGA TAT GGG CCC TTG GTG GAA GCC TCG TCA ATC CCC
AGC TCG CAG

PCR conditions were:
1 cycle at 94° C./3 minutes;
18 cycles of (94° C./1 minute, 68° C./1 minute, 72° C./2.5 minutes); and
1 cycle at 72° C./10 minutes

The DNA was then isolated from a 1% agarose gel in 1×UN-Safe TAE (Tris/acetate/EDTA) buffer (MWG-Biotech, Ebersberg, Germany) and ligated into pCRbluntII.TOPO and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with Apal and HindIII then treated with shrimp alkaline phosphatase (Roche) and gel purified.

The DLL-1 deletions cloned in pCRbluntII were cut with HindIII followed by ApaI partial restriction. The sequences were then gel purified and ligated into the pCONγ vector which was cloned into TOP10 cells.

Plasmid DNA was generated using a Qiagen Minprep kit (QIAprep™) according to the manufacturer's instructions and the identity of the PCR products was confirmed by sequencing.

The resulting construct (PCON_{χ} HDLL1 EGF1-3) coded for the following DLL-1 sequence (SEQ ID NO:33) fused to the IgG Fc domain coded by the pCONγ vector.

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPQCLHGTCQQPWQCNCQEQWGGLFCNQDLNYCTHHKPCKN
GATCTNTGQGSYTCSCRPGYTGATCELGIDE

(wherein the emboldened portion of the sequence which is single underlined is the DSL domain and the emboldened portions of the sequence which are double underlined are EGF repeats 1 to 3 respectively).

### Example 3

### Delta 1 DSL Domain Plus EGF Repeats 1-4

A human Delta 1 (DLL-1) deletion coding for the DSL domain and the first four only of the naturally occurring EGF repeats (i.e. omitting EGF repeats 5 to 8 inclusive) was generated by PCR from a DLL-1 EC domain/V5His clone using a primer pair as follows:

| | |
|---|---|
| DLac13: | |
| CACCAT GGGCAG TCGGTG CGCGCT GG | (SEQ ID NO:34) |
| and | |
| DLL1d5-8: | |
| GGTCAT GGCACT CAATTC ACAG | (SEQ ID NO:35) |

PCR conditions were:
1 cycle at 95° C./3 minutes;
18 cycles of(95° C./1 minute, 60° C./1 minute, 72° C./2.5 minutes); and
1 cycle at 72° C./10 minutes.

The DNA was then isolated from a 1% agarose gel in 1×UN-Safe TAE (Tris/acetate/EDTA) buffer (MWG-Biotech, Ebersberg, Germany) and used as a template for PCR using the following primers:

FcDL.4:

(SEQ ID NO:36)

CACCAT GGGCAG TCGGTG CGCGCT GG
and
FcDLLd5-8:

(SEQ ID NO:37)

GGATAT GGGCCC TTGGTG GAAGCG GTCATG GCACTC AATTCA
GAG

PCR conditions were:
1 cycle at 94° C./3 minutes;
18 cycles of (94° C./1 minute, 68° C./1 minute, 72° C./2.5 minutes); and
1 cycle at 72° C./10 minutes.

The fragment was ligated into pCRbluntII.TOPO and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with Apal and HindIII then treated with shrimp alkaline phosphatase (Roche) and gel purified.

The DLL-1 deletions cloned in pCRbluntII were cut with HindIII (and EcoRV to aid later selection of the desired DNA product) followed by ApaI partial restriction. The sequences were then gel purified and ligated into the pCONγ vector which was cloned into TOP10 cells.

Plasmid DNA was generated using a Qiagen Minprep kit (QIAprep™) according to the manufacturer's instructions and the identity of the PCR products was confirmed by The resulting construct (PCON_{χ} hDLL1 EGF1-4) coded for the following DLL-1 sequence (SEQ ID NO:38) fused to the IgG Fc domain coded by the pCONγ vector.

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDECDPSPCKNGGSCTDLENSY
SCTCPPGFYGKICELSAMT

(wherein the emboldened portion of the sequence which is single underlined is the DSL domain and the emboldened portions of the sequence which are double underlined are EGF repeats 1 to 4 respectively).

### Example 4

### Delta 1 DSL Domain Plus EGF Repeats 1-7

A human Delta 1 (DLL-1) deletion coding for the DSL domain and the first seven of the naturally occurring EGF repeats (i.e. omitting EGF repeat 8) was generated by PCR from a DLL-1 EC domain/V5His clone using a primer pair as follows:

| | |
|---|---|
| DLac13: | |
| CACCAT GGGCAG TCGGTG CGCGCT GG | (SEQ ID NO:39) |
| and | |
| DLL1d8: | |
| CCTGCT GACGGG GGCACT GCAGTT C | (SEQ ID NO:40) |

PCR conditions were:
1 cycle at 95° C./3 minutes;
18 cycles of (95° C./1 minute, 68° C./1 minute, 72° C./3 minutes); and
1 cycle at 72° C./10 minutes.

The DNA was then isolated from a 1% agarose gel in 1×U/V-Safe TAE (Tris/acetate/EDTA) buffer (MWG-Biotech, Ebersberg, Germany) and used as a template for PCR using the following primers:

FcDL.4:

(SEQ ID NO:41)

CACCAT GGGCAG TCGGTG CGCGCT GG
and
FCDLLd8:

(SEQ ID NO:42)

GGATAT GGGCCC TTGGTG GAAGCC CTGCTG ACGGGG GCACTG
CAGTTC

PCR conditions were:
1 cycle at 94° C./3 minutes;
18 cycles of (94° C./1 minute, 68° C./1 minute, 72° C./3minutes); and
1 cycle at 72° C./10 minutes.

The fragment was ligated into pCRbluntII.TOPO and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with Apal and HindIII then treated with shrimp alkaline phosphatase (Roche) and gel purified.

The DLL-1 deletions cloned in pCRbluntII were cut with HindIII (and EcoRV to aid later selection of the desired DNA product) followed by Apal partial restriction. The sequences were then gel purified and ligated into the pCONγ vector which was cloned into TOP10 cells.

Plasmid DNA was generated using a Qiagen Minprep kit (QIAprep™) according to the manufacturer's instructions and the PCR products were sequenced.

The resulting construct (PCON_{χ} hDLL1 EGF1-7) coded for the following DLL-1 sequence (SEQ ID NO:43) fused to the IgG Fc domain coded by the pCONγ vector.

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDECDPSPCKNGGSCTDLENSY
SCTCPPGFYGKICELSAMTCADGPCFNGGRCSDSPDGGYSCRCPVGYSGF
NCEKKIDYCSSSPCSNGAKCVDLGDAYLCRCQAGFSGRHCDDNVDDCASS
PCANGGTCRDGVNDFSCTCPPGYTGRNCSAPVSR

(wherein the emboldened portion of the sequence which is single underlined is the DSL domain and the emboldened portions of the sequence which are double underlined are EGF repeats 1 to 7 respectively).

### Example 5

### Transfection and Expression

i) Transfection and Expression of Constructs of Examples 1, 3 and 4

Cos 1 cells were separately transfected with each of the expression constructs from Examples 1, 3 and 4 above (viz pCON_{χ} HDLL1 EGF1-2, PCON_{χ} hDLL1 EGF1-4, pCON_{χ} hDLL1 EGF1-7) and pCON_{χ} control as follows:

In each case 3×10⁶ cells were plated in a 10 cm dish in Dulbecco's Modified Eagle's Medium (DMEM)+10% Fetal Calf Serum (FCS) and cells were left to adhere to the plate overnight. The cell monolayer was washed twice with 5 ml phosphate-buffered saline (PBS) and cells left in 8 ml OPTIMEM™ medium (Gibco/Invitrogen). 12 μg of the relevant construct DNA was diluted into 810 μl OPTIMEM medium and 14 μl Lipofectamine2000™ cationic lipid transfection reagent (Invitrogen) was diluted in 810 μl OPTIMEM medium. The DNA-containing and Lipofectamine2000 reagent-containing solutions were then mixed and incubated at room temperature for a minimum of 20 minutes, and then added to the cells ensuring an even distribution of the transfection mix within the dish. The cells were incubated with the transfection reagent for 6 hours before the media was removed and replaced with 20 ml DMEM+10% FCS. Supernatant containing secreted protein was collected from the cells after 5 days and dead cells suspended in the supernatant were removed by centrifugation (4,500 rpm for 5 minutes). The resulting expression products were designated: HDLL1 EGF1-2 Fc (from PCON_{χ} hDLL1 EGF1-2), hDLL1 EGF1-4 Fc (from pCON_{χ} HDLL1 EGF1-4) and hDLL1 EGF1-7 Fc (from PCON_{χ} hDLL1EGF1-7).

Expression of the Fc fusion proteins was assessed by western blot. The protein in 10 μl of supernatant was separated by 12% SDS-PAGE and blotted by semi dry apparatus on to Hybond™-ECL (Amersham Pharmacia Biotech) nitrocellulose membrane (17 V for 28 minutes). The presence of Fc fusion proteins was detected by Western blot using JDC14 anti-human IgG4 antibody diluted 1:500 in blocking solution (5% non-fat Milk solids in Tris-buffered saline with Tween 20 surfactant; TBS-T). The blot was incubated in this solution for 1 hour before being washed in TBS-T. After 3 washes of 5 minutes each, the presence of mouse anti-human IgG4 antibodies was detected using anti mouse IgG-HPRT conjugate antiserum diluted 1:10,000 in blocking solution. The blot was incubated in this solution for 1 hour before being washed in TBS-T (3 washes of 5 minutes each). The presence of Fc fusion proteins was then visualised using ECL™ detection reagent (Amersham Pharmacia Biotech).

The amount of protein present in 10 ml supernatant was assessed by comparing to Kappa chain standards containing 10 ng (7), 30 ng (8) and 100 ng (9) protein.

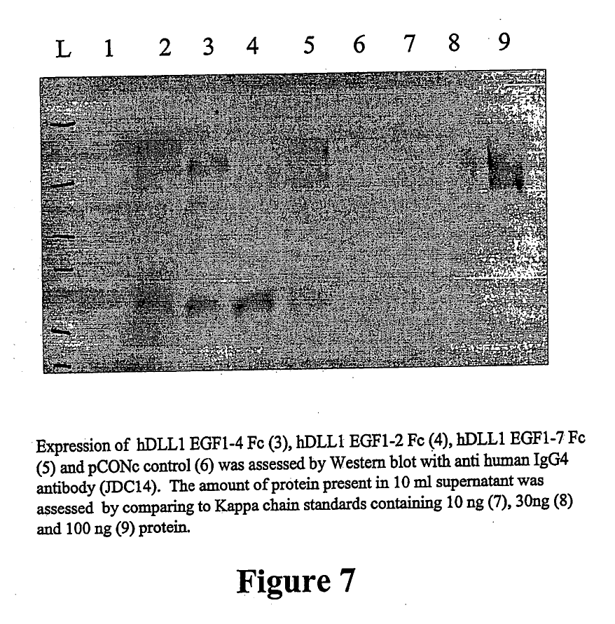

The blot results are shown in FIG. 7.

ii) Transfection and Expression of Constructs of Example 2

Cos 1 cells were transfected with the expression construct from Example 2 above (viz PCON_{χ} hDLL1 EGF1-3 as follows:

7.1×10⁵ cells were plated in a T25 flask in Dulbecco's Modified Eagle's Medium (DMEM)+10% Fetal Calf Serum (FCS) and cells were left to adhere to the plate overnight. The cell monolayer was washed twice with 5 ml phosphate-buffered saline (PBS) and cells left in 1.14 ml OPTIMEM™ medium (Gibco/Invitrogen). 2.85 μg of the relevant construct DNA was diluted into 143 μl OPTIMEM medium and 14.3 μl Lipofectamine2000™ cationic lipid transfection reagent (Invitrogen) was diluted in 129 μl OPTIMEM medium and incubated at room temperature for 45 minutes. The DNA-containing and Lipofectamine2000 reagent-containing solutions were then mixed and incubated at room temperature for 15 minutes, and then added to the cells ensuring an even distribution of the transfection mix within the flask. The cells were incubated with the transfection reagent for 18 hours before the media was removed and replaced with 3 ml DMEM+10% FCS. Supernatant containing secreted protein was collected from the cells after 4 days and dead cells suspended in the supernatant were removed by centrifugation (1,200 rpm for 5 minutes). The resulting expression product was designated: hDLL1 EGF1-3 Fc (from PCON_{χ} hDLL1 EGF1-3).

### Example 6

### Constructs for use in Notch Signalling Activity Assay

A) Construction of Luciferase Reporter Plasmid 10×CBF1-Luc (PLOR91)

An adenovirus major late promoter TATA-box motif with BglII and HindIII cohesive ends was generated as follows:

| | |
|---|---|
| BglII HindIII | |
| GATCTGGGGGGCTATAAAAGGGGGTA | (SEQ ID NO:44) |
| ACCCCCCGATATTTTCCCCCATTCGA | (SEQ ID NO:45) |

This was cloned into plasmid pGL3-Basic (Promega) between the BgiII and HindIII sites to generate plasmid pGL3-AdTATA.

A TP1 promoter sequence (TP1; equivalent to 2 CBF1 repeats) with BamH1 and BglII cohesive ends was generated as follows:

| | |
|---|---|
| BamH1 BglII | |
| 5′ GATCCCGACTCGTGGGAAAATGGGCGGAAGGGCACCGTGGGAAAATAGTA 3′ | (SEQ ID NO:46) |
| 3′ GGCTGAGCACCCTTTTACCCGCCTTCCCGTGGCACCCTTTTATCATCTAG 5′ | (SEQ ID NO:47) |

This sequence was pentamerised by repeated insertion into a BglII site and the resulting TP1 pentamer (equivalent to 10 CBF1 repeats) was inserted into pGL3-AdTATA at the BglII site to generate plasmid pLOR91.

B) Generation of a Stable CHO Cell Reporter Cell Line Expressing Full Length Notch2 and the 10×CBF1-Luc Reporter Cassette

A cDNA clone spanning the complete coding sequence of the human Notch2 gene (see, e.g. GenBank Accession No AF315356) was constructed as follows. A 3′ cDNA fragment encoding the entire intracellular domain and a portion of the extracellular domain was isolated from a human placental cDNA library (OriGene Technologies Ltd., USA) using a PCR-based screening strategy. The remaining 5′ coding sequence was isolated using a RACE (Rapid Amplification of cDNA Ends) strategy and ligated onto the existing 3′ fragment using a unique restriction site common to both fragments (Cla I). The resulting full-length cDNA was then cloned into the mammalian expression vector pcDNA3.1-V5-HisA (Invitrogen) without a stop codon to generate plasmid pLOR92. When expressed in mammalian cells, pLOR92 thus expresses the full-length human Notch2 protein with V5 and His tags at the 3′ end of the intracellular domain.

Wild-type CHO-K1 cells (e.g. see ATCC No CCL 61) were transfected with pLOR92 (pcDNA3.1-FLNotch2-V5-His) using Lipfectamine 2000™ (Invitrogen) to generate a stable CHO cell clone expressing full length human Notch2 (N2). Transfectant clones were selected in Dulbecco's Modified Eagle Medium (DMEM) plus 10% heat inactivated fetal calf serum ((HI)FCS) plus glutamine plus Penicillin-Streptomycin (P/S) plus 1 mg/ml G418 (Geneticin™—Invitrogen) in 96-well plates using limiting dilution. Individual colonies were expanded in DMEM plus 10%(HI)FCS plus glutamine plus P/S plus 0.5 mg/ml G418. Clones were tested for expression of N2 by Western blots of cell lysates using an anti-V5 monoclonal antibody (Invitrogen). Positive clones were then tested by transient transfection with the reporter vector pLOR91 (10×CBF1-Luc) and co-culture with a stable CHO cell clone (CHO-Delta) expressing full length human delta-like ligand 1 (DLL1; e.g. see GenBank Accession No AF196571). (CHO-Delta was prepared in the same way as the CHO Notch 2 clone, but with human DLL1 used in place of Notch 2. A strongly positive clone was selected by Western blots of cell lysates with anti-V5 mAb.)

One CHO-N2 stable clone, N27, was found to give high levels of induction when transiently transfected with pLOR91 (10×CBF1-Luc) and co-cultured with the stable CHO cell clone expressing full length human DLL1 (CHO-Delta1). A hygromycin gene cassette (obtainable from pcDNA3.1/hygro, Invitrogen) was inserted into pLOR91 (10×CBF1-Luc) using BamHI and SalI and this vector (10×CBF1-Luc-hygro) was transfected into the CHO-N2 stable clone (N27) using Lipfectamine 2000 (Invitrogen). Transfectant clones were selected in DMEM plus 10%(HI)FCS plus glutamine plus P/S plus 0.4 mg/ml hygromycin B (Invitrogen) plus 0.5 mg/ml G418 (Invitrogen) in 96-well plates using limiting dilution. Individual colonies were expanded in DMEM plus 10%(HI)FCS plus glutamine plus P/S+0.2 mg/ml hygromycin B plus 0.5 mg/ml G418 (Invitrogen).

Clones were tested by co-culture with a stable CHO cell clone expressing FL human DLL1. Three stable reporter cell lines were produced N27#11, N27#17 and N27#36. N27#11 was selected for further use because of its low background signal in the absence of Notch signalling, and hence high fold induction when signalling is initiated. Assays were set up in 96-well plates with 2×10⁴ N27#11 cells per well in 100 μl per well of DMEM plus 10%(HI)FCS plus glutamine plus P/S.

### Example 7

### Luciferase Assay for Detecting Notch Signalling

The Fc-tagged Notch ligand expression products from Example 5 (hDLL1 EGF1-2 Fc, hDLL1 EGF1-4 Fc and HDLL1 EGF1-7 Fc) were each separately immobilised on Streptavidin-Dynabeads (CELLection Biotin Binder Dynabeads [Cat. No. 115.21] at 4.0×10⁸ beads/ml from Dynal (UK) Ltd; “beads”) in combination with biotinylated α-IgG-4 (clone JDC14 at 0.5 mg/ml from Pharmingen [Cat. No. 555879]) as follows:

1×10⁷ beads (25 μl of beads at 4.0×10⁸ beads/ml) and 2 μg biotinylated α-IgG-4 was used for each sample assayed. PBS was added to the beads to 1 ml and the mixture was spun down at 13,000 rpm for 1 minute. Following washing with a further 1 ml of PBS the mixture was spun down again. The beads were then resuspended in a final volume of 100 μl of PBS containing the biotinylated α-IgG-4 in a sterile Eppendorf tube and placed on shaker at room temperature for 30 minutes. PBS to was added to 1 ml and the mixture was spun down at 13,000 rpm for 1 minute and then washed twice more with 1 ml of PBS.

The mixture was then spun down at 13,000 rpm for 1 minute and the beads were resupsended in 50 μl PBS per sample. 50 μl of biotinylated α-IgG-4-coated beads were added to each sample and the mixture was incubated on a rotary shaker at 4° C. overnight. The tube was then spun at 1000 rpm for 5 minutes at room temperature.

The beads then were washed with 10 ml of PBS, spun down, resupended in 1 ml of PBS, transferred to a sterile Eppendorf tube, washed with a further 2×1 ml of PBS, spun down and resuspended in a final volume of 100 μl of DMEM plus 10%(HI)FCS plus glutamine plus P/S, i.e. at 1.0×10⁵ beads/μl.

Stable N27#1 1 cells (T₈₀ flask)were removed using 0.02% EDTA solution (Sigma), spun down and resuspended in 10 ml DMEM plus 10%(HI)FCS plus glutamine plus P/S. 10 μl of cells were counted and the cell density was adjusted to 1.0×10⁵ cells/ml with fresh DMEM plus 10%(HI)FCS plus glutamine plus P/S. 1.0×10⁵ of the cells were plated out per well of a 24-well plate in a 1 ml volume of DMEM plus 10%(HI)FCS plus glutamine plus P/S and cells were placed in an incubator to settle down for at least 30 minutes.

20 μl of beads were then added in duplicate to a pair of wells to give 2.0×10⁶ beads/well (100 beads/cell). The plate was left in a CO₂ incubator overnight.

Supernatant was then removed from all the wells, 100 μl of SteadyGlo™ luciferase assay reagent (Promega) was added and the resulting mixture left at room temperature for 5 minutes.

The mixture was then pipetted up and down 2 times to ensure cell lysis and the contents from each well were transferred to a 96 well plate (with V-shaped wells) and spun in a plate holder for 5 minutes at 1000 rpm at room temperature.

175 μl of cleared supernatant was then transferred to a white 96-well plate (Nunc) leaving the beads pellet behind.

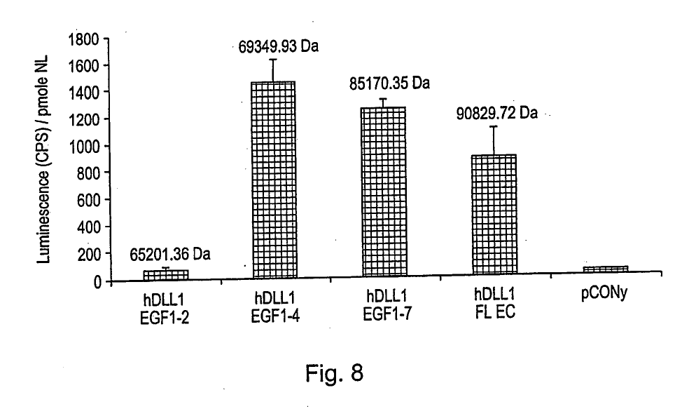

Luminescence was then read in a TopCount™ (Packard) counter. Results are shown in FIG. 8 (where activity from a construct comprising a full Dll1 EC domain is also shown for comparison).

### Example 8

### Jagged Truncations

Nucleotide sequences coding for the human Jagged1 (hJag1) DSL domain and the first two, three, four and sixteen respectively of the naturally occurring Jagged EGF repeats were generated by PCR from a human Jagged-1 (see e.g. GenBank Accession No U61276) cDNA. The sequences were then purified, ligated into a pCONγ expression vector coding for an immunogolbulin Fc domain, expressed and coated onto microbeads. The expressed proteins comprised the DSL domain and the first two (hJag1 EGF1-2), three (hJag1 EGF1-3), four (hJag1 EGF1-4) and sixteen (hJag1 EGF1-16) respectively of the Jagged EGF repeats fused to the IgG Fc domain encoded by the pCONγ vector.

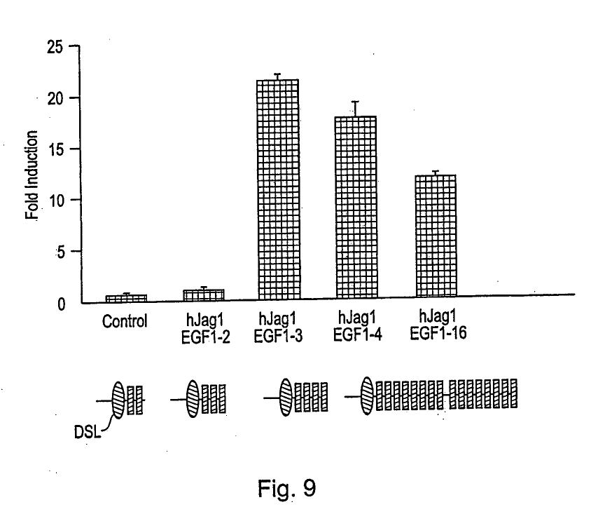

Beads coated with each of the expressed proteins were then tested for activity in the Notch signalling reporter assay as described above. The activity data obtained is shown in FIG. 9.

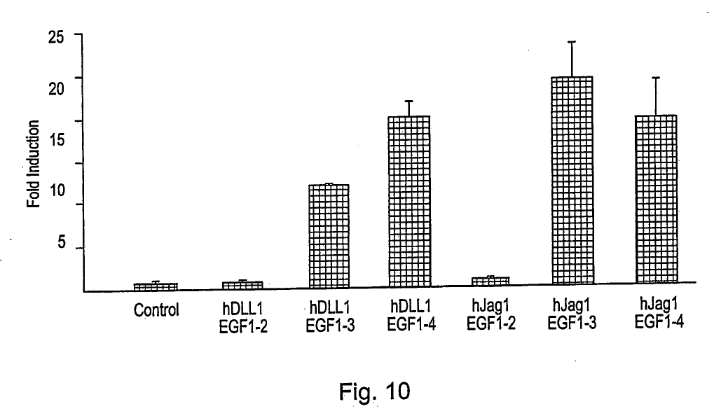

Similar assays were conducted with expressed Jagged proteins from this Example alongside corresponding Delta proteins from Example 5, for more ready comparison. Results are shown in FIG. 10.

### Example 9

### Assay of Jagged EGF1-2 with Increased Sensitivity

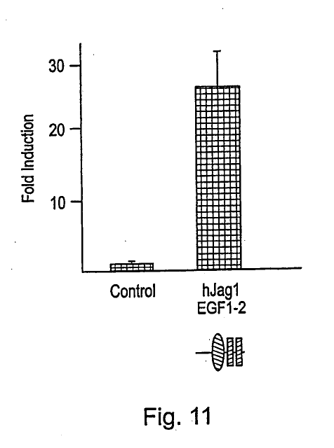

In a further experiment purified protein comprising human Jagged1 DSL domain plus the first two EGF repeats (hJag1EGF1-2) from Example 7 was coated onto beads and tested for activity in a Notch reporter assay as described above, at a higher protein load, to give greater sensitivity. The activity data obtained is shown in FIG. 11.

### Example 10

### Soluble hJagged1[2EGF]-Fc Antagonizes Notch Activation in CHO—N2 Cells

i) Preparation of hJagged1[2EGF]-Fc

A fusion protein comprising a truncated extracellular domain of human Jagged1 (up to the end of the second EGF-like domain) fused to the Fc domain of human IgG4 (“hJagged1[2EGF]-Fc”) was prepared by inserting corresponding Jagged1 cDNA into the expression vector pCONγ (Lonza Biologics, Slough, UK) and expressing the resulting construct in CHO cells.

ii) Antagonist Assays of Notch Signalling from hDLL1-Fc-Coated Dynabeads

A volume of Dynabeads beads corresponding to the total number required was removed from a stock of beads at 4.0×10⁸ beads/ml. This was washed twice with 1 ml of PBS, and resuspended in a final volume of 100 μl of PBS containing the biotinylated anti-IgG-4 antibody (clone JDC14 at 0.5 mg/ml from Pharmingen [Cat. No. 555879]) in a sterile Eppendorf tube and placed on shaker at room temperature for 30 minutes. The amount of biotinylated anti-IgG4 antibody needed to coat the beads was galculated relative to the fact that 1×10⁷ streptavidin Dynabeads bind a maximum of 2 μg of antibody.

After coating the beads with antibody they were washed with 3 times with 1 ml of PBS and finally resuspended in hDelta1-Fc purified protein diluted in PBS. The amount of hDelta1-Fc used to coat the beads was calculated from the result of an experiment in which a dilution series of hDLL1-Fc concentrations was set up with 1×10⁷ anti-IgG4-coated beads and it was found that 2-5 μg of hDelta1-Fc was enough protein to coat 1×10⁷ anti-IgG4-coated beads in a 1 ml volume of PBS and give a good signal when added to the reporter cells. So usually 5 μg of hDelta1-Fc protein was added per 10⁷ beads to be coated and the ligand was allowed to bind to the beads in a 1 ml volume for 2 h at room temperature (or 4° C. overnight) on a rotary shaker to keep the beads in suspension. After coating the beads with hDelta1-Fc the beads were washed 3 times with 1 ml of PBS and finally resuspended complete DMEM at 2×10⁷ beads per ml so that addition of 100 μl of this to a well of 2×10⁴ reporter cells gave a ratio of 100 beads:cell.

To set up the bead antagonist assay, N27#11 cells (T₈₀ flask) were removed using 0.02% EDTA solution (Sigma), spun down and resuspended in 10 ml DMEM plus 10%(HI)FCS plus glutamine plus P/S. Ten μl of cells were counted and the cell density was adjusted to 2.0×10⁵ cells/ml with fresh DMEM plus 10%(HI)FCS plus glutamine plus P/S.

The reporter cells were plated out at 100 μl per well of a 96-well plate (i.e. 2×10⁴ cells per well) and were placed in an incubator to settle down for at least 30 minutes.

Purified soluble ligands—either hJagged1 [2EGF]-Fc or hDelta1-Fc were diluted in complete DMEM to 5× final concentration required in the assay and 50 μl of diluted ligand was added to the 100 μl of N27#11 cells in a 96-well plate. Then 100 μl of hDelta1-Fc-Dynabeads at 2×10⁷ beads/ml was added to initiate the signalling—giving a final volume of 250 μl in each well. The plate was then placed at 37° C. in an incubator overnight.

The following day 150 μl of supernatant was then removed from all the wells, 100 μl of SteadyGlo™ luciferase assay reagent (Promega) was added and the resulting mixture left at room temperature for 5 minutes. The mixture was then pipetted up and down 2 times to ensure cell lysis and the contents from each well were transferred to a 96 well plate (with V-shaped wells) and spun in a plate holder for 5 minutes at 1000 rpm at room temperature. The cleared supernatant was then transferred to a white 96-well plate (Nunc) leaving the beads pellet behind. Luminescence was then read in a TopCount™ (Packard) counter.

iii) Antagonist Assay of Notch Signalling from CHO-Delta Cells

CHO-Delta cells (as described above) were maintained in DMEM plus 10% (HI)FCS plus glutamine plus P/S plus 0.5 mg/ml G418. Just prior to use the cells were removed from a T80 flask using 0.02% EDTA solution (Sigma), spun down and resuspended in 10 ml DMEM plus 10%(HI)FCS plus glutamine plus P/S. 10 μl of cells were counted and the cell density was adjusted to 5.0×10⁵ cells/ml with fresh DMEM plus 10%(HI)FCS plus glutamine plus P/S.

To set up the CHO-Delta antagonist assay, N27#11 cells (T₈₀ flask) were removed using 0.02% EDTA solution (Sigma), spun down and resuspended in 10 ml DMEM plus 10%(HI)FCS plus glutamine plus P/S. 10 μl of cells were counted and the cell density was adjusted to 2.0×10⁵ cells/ml with fresh DMEM plus 10%(HI)FCS plus glutamine plus P/S. The reporter cells were plated out at 100 μl per well of a 96-well plate (i.e. 2×10⁴ cells per well) and were placed in an incubator to settle down for at least 30 minutes.

Purified soluble ligands—either hJagged1[2EGF]-Fc or hDelta1-Fc were diluted in complete DMEM to 5× final concentration required in the assay and 50 μl of diluted ligand was added to the 100 μl of N27#11 cells in a 96-well plate. Then 100 μl of CHO-Delta cells at 5×10⁵ cells/ml was added to initiate the signalling—giving a final volume of 250 μl in each well. The plate was then placed at 37° C. in an incubator overnight.

The following day 150 μl of supernatant was then removed from all the wells, 100 μl of SteadyGlo™ luciferase assay reagent (Promega) was added and the resulting mixture left at room temperature for 5 minutes. The mixture was then pipetted up and down 2 times to ensure cell lysis and the contents from each well were transferred to a white 96-well plate (Nunc). Luminescence was then read in a TopCount™ (Packard) counter.

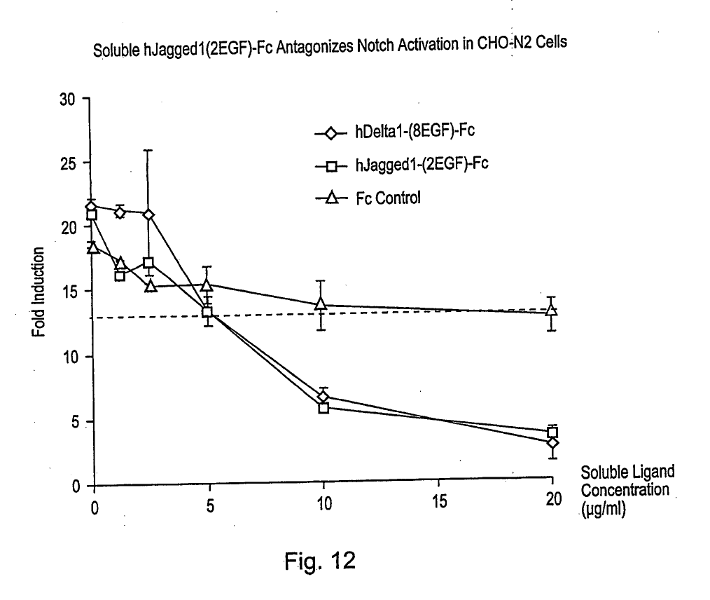

Results are shown in FIG. 12. It can be seen that the truncated Jagged protein with just 2 EGF repeats (hJagged1[2EGF]-Fc) provided substantially the same inhibition of Notch signalling as a corresponding protein comprising a full length human Delta1extracellular domain (hDelta1-Fc).

### Example 11

### ELISA Assay Method for Detecting Notch Signalling Activity

(i) CD4+ Cell Purification

Spleens were removed from mice (variously Balb/c females, 8-10 weeks, C57B/6 females, 8-10 weeks, D011.10 transgenic females, 8-10 weeks) and passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHCO₃, 0.1 mM Na₂EDTA in double distilled water) per spleen (to lyse red blood cells). The cells were then washed once with RIOF medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

(ii) Antibody Coating

The following protocols were used for coating 96 well flat-bottomed plates with antibodies.

A) The plates were coated with Dulbecco's Phosphate Buffered Saline (DPBS) plus 1 μg/ml anti-CD3 antibody (Pharmingen, San Diego, US: Cat No 553058, Clone No 145-2C11) plus 1 μg/ml anti-IgG4 antibody (Pharmingen Cat No 555878). 100 μl of coating mixture was used per well. Plates were incubated overnight at 4° C. then washed with DPBS. Each well then received either 100 μl DPBS or 100 μl DPBS plus 10 μg/ml hDelta1-Fc.

The plates were incubated for 2-3 hours at 37° C. then washed again with DPBS before cells (prepared as described above) were added.

B) Alternatively, the plates were coated with DPBS plus 1 μg/ml anti-hamsterIgG antibody (Pharmingen Cat No 554007) plus 1 μg/ml anti-IgG4 antibody. 100 μl of coating mixture was added per well. Plates were incubated overnight at 4° C. then washed with DPBS. Each well then received either 100 μl DPBS plus anti-CD3 antibody (1 μg/ml) or, 100 μl DPBS plus anti-CD3 antibody (1 μg/ml) plus hDelta1-Fc (10 μg/ml). The plates were incubated for 2-3 hours at 37° C. then washed again with DPBS before cells (prepared as described above) were added.

(iii) Primary Polyclonal Stimulation and ELISA

CD4+ cells were cultured in 96 well, flat-bottomed plates pre-coated according to protocol A or B above. Cells were re-suspended, following counting, at 2×10⁶/ml in R10F medium plus 4 μg/ml anti-CD28 antibody (Pharmingen, Cat No 553294, Clone No 37.51). 100 μl cell suspension was added per well. 100 μl of RIOF medium was then added to each well to give a final volume of 200 μl (2×10⁵ cells/well, anti-CD28 final concentration 2 μg/ml) The plates were then incubated at 37° C. for 72 hours.

125 μl supernatant was then removed from each well and stored at −20° C. until tested by ELISA for IL-10, IFNg and IL-13 using antibody pairs from R & D Systems (Abingdon, UK). The cells were then split 1 in 3 into new wells (not coated) and fed with R10F medium plus recombinant human IL-2 (2.5 ng/ml, PeproTech Inc, London, UK: Cat No 200-02).

### Example 12

i) Preparation of Modulator of Notch Signalling in Form of Notch Ligand Extracellular Domain Fragment with Free Cysteine Tail for Polymer Coupling

A protein fragment comprising amino acids 1 to 332 (i.e. comprising DSL domain plus first 3 EGF repeats) of human Delta 1 (DLL-1; for sequence see GenBank Accession No AF003522) and ending with a free cysteine residue (“D1E3Cys”) was prepared as follows:

A template containing the entire coding sequence for the extracellular (EC) domain of human DLL-1 (with two silent mutations) was prepared by a PCR cloning strategy from a placental cDNA library made from placental polyA+RNA (Clontech; cat no 6518-1) and combined with a C-terminal V5HIS tag in a pCDNA3.1 plasmid (Invitrogen, UK) The template was cut HindIII to PmeI to provide a fragment coding for the EC domain and this was used as a template for PCR using primers as follows:

5′-primer:

(SEQ ID NO:48)

CAC CAT GGG CAG TCG GTG CGC GCT GG
3′-primer:

(SEQ ID NO:49)

GTC TAC GTT TAA ACT TAA CAC TCG TCA ATC CCC AGC
TCG CAG GTG

PCR was carried out using Pfu turbo polymerase (Stratagene, La Jolla, Calif., US) with cycling conditions as follows: 95C 5 min, 95C 1 min, 45-69C 1 min, 72C 1 min for 25 cycles, 72C 10 min.

The products at 58C, 62C & 67C were purified from 1% agarose gel in 1×TAE using a Qiagen gel extraction kit according to the manufacturer's instructions, ligated into pCRIIblunt vector (InVitrogen TOPO-blunt kit) and then transformed into TOP10 cells (InVitrogen). The resulting clone sequence was verified, and only the original two silent mutations were found to be present in the parental clone.

The resulting sequence coding for “D1E3Cys” was excised using PmeI and HindIII, purified on 1% agarose gel, 1× TAE using a Qiagen gel extraction kit and ligated into pCDNA3.1V5HIS (Invitrogen) between the PmeI and HindIII sites, thereby eliminating the V5HIS sequence. The resulting DNA was transformed into TOP10 cells. The resulting clone sequence was verified at the 3′-ligation site.

The D1E3Cys-coding fragment was excised from the pCDNA3.1 plasmid using PmeI and HindIII. A pEE14.4 vector plasmid (Lonza Biologics, UK) was then restricted using EcoRI, and the 5′-overhangs were filled in using Klenow fragment polymerase. The vector DNA was cleaned on a Qiagen PCR purification column, restricted using HindIII, then treated with Shrimp Alkaline Phosphatase (Roche). The pEE14.4 vector and D1E3cys fragments were purified on 1% agarose gel in 1× TAE using a Qiagen gel extraction kit prior to ligation (T4 ligase) to give plasmid pEE14.4 DLLA4-8cys. The resulting clone sequence was verified.

The D1E3Cys coding sequence is as follows (SEQ ID NO:50):

| | |
|---|---|
| 1 | atgggcagtc ggtgcgcgct ggccctggcg gtgctctcgg ccttgctgtg |
| 51 | tcaggtctgg agctctgggg tgttcgaact gaagctgcag gagttcgtca |
| 101 | acaagaaggg gctgctgggg aaccgcaact gctgccgcgg gggcgcgggg |
| 151 | ccaccgccgt gcgcctgccg gaccttcttc cgcgtgtgcc tcaagcacta |
| 201 | ccaggccagc gtgtcccccg agccgccctg cacctacggc agcgccgtca |
| 251 | cccccgtgct gggcgtcgac tccttcagtc tgcccgacgg cgggggcgcc |
| 301 | gactccgcgt tcagcaaccc catccgcttc cccttcggct tcacctggcc |
| 351 | gggcaccttc tctctgatta ttgaagctct ccacacagat tctcctgatg |
| 401 | acctcgcaac agaaaaccca gaaagactca tcagccgcct ggccacccag |
| 451 | aggcacctga cggtgggcga ggagtggtcc caggacctgc acagcagcgg |
| 501 | ccgcacggac ctcaagtact cctaccgctt cgtgtgtgac gaacactact |
| 551 | acggagaggg ctgctccgtt ttctgccgtc cccgggacga tgccttcggc |
| 601 | cacttcacct gtggggagcg tggggagaaa gtgtgcaacc ctggctggaa |
| 651 | agggccctac tgcacagagc cgatctgcct gcctggatgt gatgagcagc |
| 701 | atggattttg tgacaaacca ggggaatgca agtgcagagt gggctggcag |
| 751 | ggccggtact gtgacgagtg tatccgctat ccaggctgtc tccatggcac |
| 801 | ctgccagcag ccctggcagt gcaactgcca ggaaggctgg gggggccttt |
| 851 | tctgcaacca ggacctgaac tactgcacac accataagcc ctgcaagaat |
| 901 | ggagccacct gcaccaacac gggccagggg agctacactt gctcttgccg |
| 951 | gcctgggtac acaggtgcca cctgcgagct ggggattgac gagtgttaa |

The DNA was prepared for stable cell line transfection/selection in a Lonza GS system using a Qiagen endofree maxi-prep kit.

ii) Expression of D1E3Cys

Linearisation of DNA

The pEE14.4 DLLA4-8cys plasmid DNA from (i) above was linearised by restriction enzyme digestion with PvuI, and then cleaned up using phenol chloroform isoamyl alcohol (LkA), followed by ethanol precipitation. Plasmid DNA was checked on an agarose gel for linearisation, and spec'd at 260/280 nm for quantity and quality of prep.

Transfection

CHO-K1 cells were seeded into 6 wells at 7.5×10⁵ cells per well in 3 ml media (DMEM 10% FCS) 24hrs prior to transfection, giving 95% confluency on the day of transfection.

Lipofectamine 2000 was used to transfect the cells using 5 ug of linearised DNA. The transfection mix was left on the cell sheet for 5½ hours before replacing with 3 ml semi-selective media (DMEM, 10% dFCS, GS) for overnight incubation.

At 24 hours post-transfection the media was changed to full selective media (DMEM (Dulbecco's Modified Eagle Medium), 10%dFCS (fetal calf serum), GS (glutamine synthase), 25 uM L-MSX (methionine sulphoximine)) and incubated further.

Cells were plated into 96 wells at 10⁵ cells per well on days 4 and 15 after transfection.

96 well plates were screened under a microscope for growth 2 weeks post clonal plating. Single colonies were identified and scored for % confluency. When colony size was >30% media was removed and screened for expression by dot blot against anti-human-Delta-1 antisera. High positives were confirmed by the presence of a 36 kDa band reactive to anti-human-Delta-1 antisera in PAGE Western blot of media.

Cells were expanded by passaging from 96 well to 6 well to T25 flask before freezing.

The fastest growing positive clone (LC09 0001) was expanded for protein expression.

D1E3Cys Expression and Purification

T500 flasks were seeded with 1×10⁷ cells in 80 ml of selective media. After 4 days incubation the media was removed, cell sheet rinsed with DPBS and 150 ml of 325 media with GS supplement added to each flask. Flasks were incubated for 7 further days before harvesting. Harvest media was filtered through a 0.65-0.45 um filter to clarify prior to freezing. Frozen harvests were purified by FPLC as follows:

Frozen harvest was thawed and filtered. A 17 ml Q Sepharose column was equilibrated in 0.1M Tris pH8 buffer, for 10 column volumes. The harvest was loaded onto the column using a P1 pump set at 3 ml/min, the flowthrough was collected into a separate container (this is a reverse purification—a lot of the BSA contaminant binds to the Q Sepharose FF and our target protein does not and hence remains in the flowthrough). The flowthrough was concentrated in a TFF rig using a 10 kDa cut off filter cartridge, during concentration it was washed 3× with 0.1M Sodium phosphate pH 7 buffer. The 500 ml was concentrated down to 35 ml, to a final concentration of 3 mg/ml.

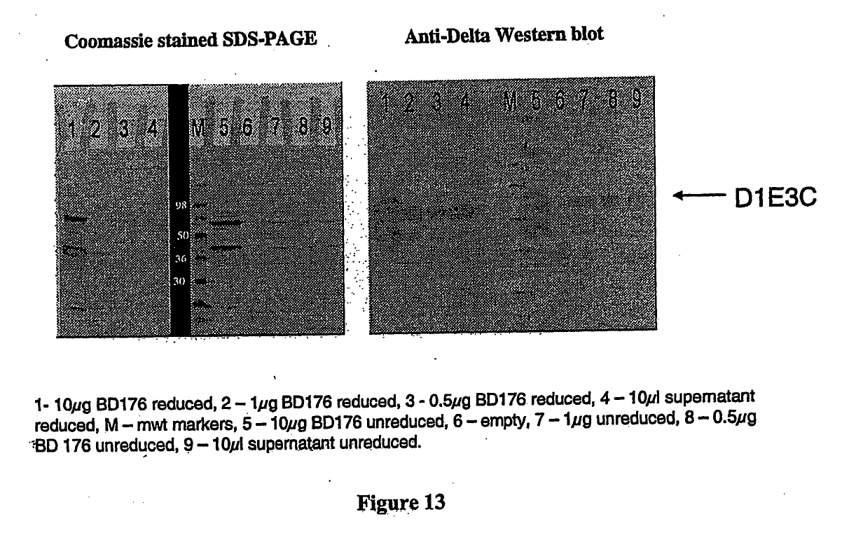

Samples were run on SDS PAGE reduced and non-reduced (gels are shown in FIG. 13)

The amino acid sequence of the resulting expressed D1E3Cys protein was as follows (SEQ ID NO:51):

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDEC

(wherein the sequence in italics is the leader peptide, the underlined sequence is the DSL domain, the bold sequences are the three EGF repeats, and the terminal Cys residue is shown bold underlined).
iii) Reduction of D1E3cys Protein

40 μg D1E3Cys protein from (ii) above was made up to 100 μl to include 100 mM sodium phosphate pH 7.0 and 5 mM EDTA. 2 volumes of immobilised TCEP (tris[2-carboxyethyl]phosphine hydrochloride; Pierce, Rockford, Ill., US, Cat No: 77712; previously washed 3 times 1 ml 100 mM sodium phosphate pH 7.0) were added and the mixture was incubated for 30 minutes at room temperature, with rotating.

The resin was pelleted at room temperature in a microfuge (13,000 revs/min, 5 minutes) and the supernatant was transferred to a clean Eppendorf tube and stored on ice. Protein concentration was measured by Warburg-Christian method.

This fragment is linked to a polymer such as dextran or PEG as described above to provide the final conjugate.

### Example 13

### Coupling of D1E3cys to Amino-Dextran to Provide Conjugate

i) Purification of Expressed D1E3CYs by HIC

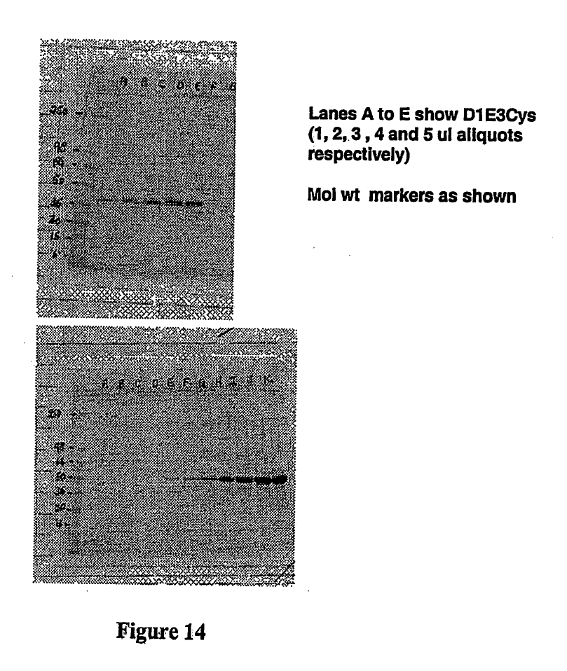

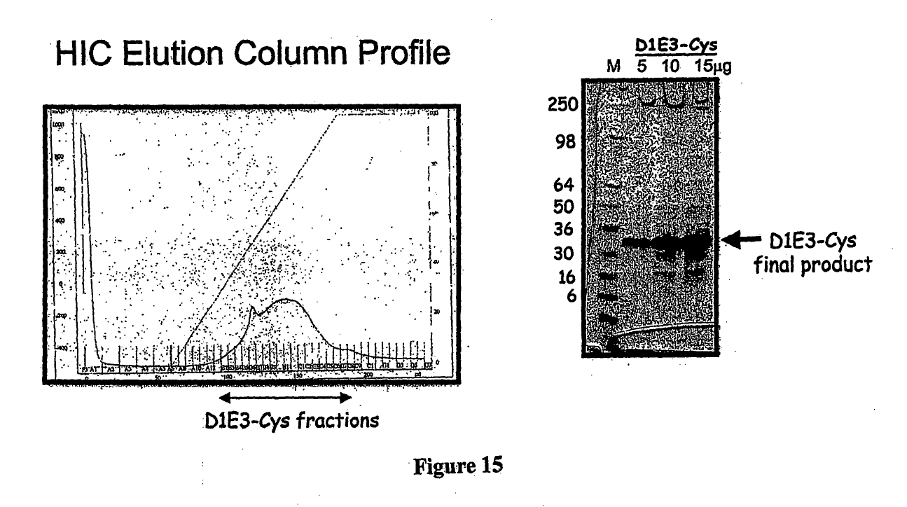

Harvests from Example 12 above were purified using Hydrophobic Interaction Chromatography (HIC), the eluate was then concentrated and buffer exchanged using centrifugal concentrators according to the manufacturers' instructions. The purity of the product was determined by SDS PAGE. Sample gels are shown in FIG. 14 and a sample gel and purification trace is shown in FIG. 15.

ii) Maleimide Substitution of Amino-Dextran (Polymer Activation)

Amino-dextran of molecular mass 500,000 Da (dextran, amino, 98 moles amine/mole; Molecular Probes, ref D-7144), 3.2 mg/ml, was derivatised/activated with sulfo-SMCC (sulfosuccinimidyl 4-[N-maleimidomethyl]-cyclohexane-1-carboxylate; Pierce, ref 22322) at 73 moles sulfo-SMCC per mole amino-dextran in 100 mM sodium phosphate pH8.0 for 1 h, 22° C.

The amino content of the dextran and the level of maleimide substitution was measured using a Ninhydrin assay. Aliquots of dextran derivative or B-alanine (Sigma, A-7752) were made to 50 μl in 100 mM sodium phosphate pH7.0 and diluted in water to 250 μl. Ninhydrin reagent solution (Sigma, N1632) was added, 1 vol., and samples heated 100° C., 15 min. After cooling on ice 1 vol. 50% ethanol was added, mixed and the solution clarified by centrifugation. Absorbance was recorded at 570 nm.

The resulting maleimido-dextran was purified and concentrated by buffer exchange using Vivaspin 6 ml concentrators (VivaScience, VS0612) and 3×5 ml, 100 mM sodium phosphate pH7.0.

The concentration of dextran was measured using an ethanol precipitation/turbidity assay. Aliqouts of dextran derivative were made to 50 μl in 100 mM sodium phosphate pH7.0. Water was added to make 500 μl final volume, dextran was precipitated by the addition of 1 vol. absolute ethanol and absorbance was recorded at 600 nm.

iii) Partial Reduction of D1E3cys

D1E3cys protein (purified as in (i) above) at 1 mg/ml in 100 mM sodium phosphate pH7.0 was reduced using TCEP.HCl (Tris(2-carboxyethyl)phosphine hydrochloride; Pierce, 20490) at a 10-fold molar excess of reducing agent for 1h at 22° C. The protein was purified by buffer exchange using Sephadex G-25, PD-10 columns (Amersham biosciences, 17-0851-01) into 100 mM sodium phosphate pH7.0 followed by concentration in Vivaspin 6 ml concentrators. Protein concentration was estimated using the Warburg-Christian A280/A260 method.

The efficiency of reduction can be estimated using the Ellman's assay. The supplied D1E3cys protein has no free thiol groups, whereas partially reduced D1E3cys is predicted to have a single free thiol group per mole of protein. Using a 96-well microtitre plate, aliqouts of D1E3cys protein or L-cysteine hydrochloride (Sigma, C-1276) were made to 196 ul in 100 mM sodium phosphate pH7.0 and 4 ul 4 mg/ml Ellman's reagent (in 100 mM sodium phosphate pH 7.0) was added. Reactions were incubated for 15 min at 22° C. and absorbance was recorded at 405 nm.

iv) Coupling of Reduced D1E3cys to Maleimido-Dextran.

The derivatized maleimido-dextran was added to concentrated, reduced D1E3cys at a 1:75 molar ratio of dextran to D1E3cys. Coupling proceeded for 18h, 4 ° C.

The resulting D1E3cys-dextran polymer (D1E3Cys-dextran conjugate; comprising aminodextrans each coupled to a large number of D1E3Cys proteins via SMCC linkers) was purified by gel permeation chromatography using a Superdex 200 (Amersham Biosciences, 17-1043-10) column attached to an AKTA purifier FPLC (Amersham Biosciences) in 100 mM sodium phosphate pH7.0. At a flow rate of 1 ml/min, 1 ml fractions were collected. The protein complex was then concentrated in Vivaspin 6 ml concentrators and protein concentration was measured using the Warburg-Christian A280/A260 method.

The invention is additionally described by the following numbered paragraphs:

1. A pharmaceutical composition comprising a Notch ligand protein or polypeptide consisting essentially of the following components: i) a Notch ligand DSL domain; ii) 1 to 5 EGF repeat domains; iii) optionally all or part of a Notch ligand N-terminal domain; and iv) optionally one or more heterologous amino acid sequences;or a multimer of such a protein or polypeptide (wherein each monomer may be the same or different); or a polynucleotide coding for such a Notch ligand protein or polypeptide; in combination with a pharmaceutically acceptable carrier.

2. A pharmaceutical composition comprising a Notch ligand protein or polypeptide comprising: i) a Notch ligand DSL domain; ii) 1 to 5 (but no more than 5) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide;in combination with a pharmaceutically acceptable carrier.

3. A pharmaceutical composition comprising a Notch ligand protein or polypeptide consisting essentially of the following components: i) a Notch ligand DSL domain; ii) 1 to 4 EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; in combination with a pharmaceutically acceptable carrier.

4. A pharmaceutical composition comprising a Notch ligand protein or polypeptide comprising: i) a Notch ligand DSL domain; ii) 1 to 4 (but no more than 4) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; in combination with a pharmaceutically acceptable carrier.

5. A pharmaceutical composition comprising a Notch ligand protein or polypeptide comprising: i) a Notch ligand DSL domain; ii) 1 (but no more than 1) EGF repeat domain; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; in combination with a pharmaceutically acceptable carrier.

6. A pharmaceutical composition comprising a Notch ligand protein or polypeptide comprising: i) a Notch ligand DSL domain; ii) 2 (but no more than 2 ) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; in combination with a pharmaceutically acceptable carrier.

7. A pharmaceutical composition comprising a Notch ligand protein or polypeptide comprising: i) a Notch ligand DSL domain; ii) 3 (but no more than 3) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; in combination with a pharmaceutically acceptable carrier.

8. A pharmaceutical composition comprising a Notch ligand protein or polypeptide comprising: i) a Notch ligand DSL domain; ii) 4 (but no more than 4) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; in combination with a pharmaceutically acceptable carrier.

9. A pharmaceutical composition comprising a Notch ligand protein or polypeptide comprising: i) a Notch ligand DSL domain; ii) 5 (but no more than 5) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; in combination with a pharmaceutically acceptable carrier.

10. A composition as described in any one of the preceding paragraphs wherein the Notch ligand protein or polypeptide activates a human Notch receptor.

11. A composition as described in any one of the preceding paragraphs wherein the heterologous amino acid sequence comprises or codes for all or part of an immunoglobulin Fc domain.

12. A composition as described in any one of the preceding paragraphs wherein the Notch ligand protein, polypeptide or polynucleotide comprises or codes for at least part of a mammalian Notch ligand sequence.

13. A composition as described in any one of the preceding paragraphs wherein the Notch ligand protein, polypeptide or polynucleotide comprises or codes for at least part of a human Notch ligand sequence.

14. A composition as described in any one of the preceding paragraphs wherein the Notch ligand protein, polypeptide or polynucleotide comprises or codes for Notch ligand domains from Delta, Serrate or Jagged or domains having at least 30% amino acid sequence similarity thereto.

15. A composition as described in any one of the preceding paragraphs wherein the Notch ligand protein, polypeptide or polynucleotide comprises or codes for Notch ligand domains from Delta1, Delta 3, Delta 4, Jagged 1 or Jagged 2 or domains having at least 30% amino acid sequence similarity thereto.

16. A multimeric Notch ligand protein or polypeptide comprising monomers consisting essentially of the following components: i) a Notch ligand DSL domain; ii) 1 to 5 EGF repeat domains; iii) optionally all or part of a Notch ligand N-terminal domain; and iv) optionally one or more heterologous amino acid sequences; wherein each monomer may be the same or different.

17. A multimeric Notch ligand protein or polypeptide comprising monomers comprising: i) a Notch ligand DSL domain; ii) 1 to 5 (but no more than 5) EGF repeat domains; iii) optionally all or part of a Notch ligand N-terminal domain; and iv) optionally one or more heterologous amino acid sequences; wherein each monomer may be the same or different.

18. A multimeric Notch ligand protein or polypeptide comprising monomers consisting essentially of: i) a Notch ligand DSL domain; ii) 1 to 4 EGF repeat domains; iii) optionally all or part of a Notch ligand N-terminal domain; and iv) optionally one or more heterologous amino acid sequences; wherein each monomer may be the same or different.

19. A multimeric Notch ligand protein or polypeptide comprising monomers comprising: i) a Notch ligand DSL domain; ii) 1 to 4 (but no more than 4) EGF repeat domains; iii) optionally all or part of a Notch ligand N-terminal domain; and iv) optionally one or more heterologous amino acid sequences; wherein each monomer may be the same or different.

20. A multimeric Notch ligand protein or polypeptide comprising monomers comprising: i) a Notch ligand DSL domain; ii) 1 (but no more than 1) EGF repeat domain; iii) optionally all or part of a Notch ligand N-terminal domain; and iv) optionally one or more heterologous amino acid sequences; wherein each monomer may be the same or different.

21. A multimeric Notch ligand protein or polypeptide comprising monomers comprising: i) a Notch ligand DSL domain; ii) 2 (but no more than 2) EGF repeat domains; iii) optionally all or part of a Notch ligand N-terminal domain; and iv) optionally one or more heterologous amino acid sequences; wherein each monomer may be the same or different.

22. A multimeric Notch ligand protein or polypeptide comprising monomers comprising: i) a Notch ligand DSL domain; ii) 3 (but no more than 3) EGF repeat domains; iii) optionally all or part of a Notch ligand N-terminal domain; and iv) optionally one or more heterologous amino acid sequences; wherein each monomer may be the same or different.

23. A multimeric Notch ligand protein or polypeptide comprising monomers comprising: i) a Notch ligand DSL domain; ii) 4 (but no more than 4) EGF repeat domains; iii) optionally all or part of a Notch ligand N-terminal domain; and iv) optionally one or more heterologous amino acid sequences; wherein each monomer may be the same or different.

24. A multimeric Notch ligand protein or polypeptide comprising monomers comprising: i) a Notch ligand DSL domain; ii) 5 (but no more than 5) EGF repeat domains; iii) optionally all or part of a Notch ligand N-terminal domain; and iv) optionally one or more heterologous amino acid sequences; wherein each monomer may be the same or different.

25. A Notch ligand protein or polypeptide which consists essentially of the following components: i) a Notch ligand DSL domain; ii) 1 to 3 Notch ligand EGF domains; iii) optionally all or part of a Notch ligand N-terminal domain; and iv) optionally one or more heterologous amino acid sequences; or a polynucleotide which codes for such a Notch ligand protein or polypeptide.

26. A Notch ligand protein or polypeptide which consists essentially of the following components: i) a Notch ligand DSL domain; ii) one Notch ligand EGF domain; iii) optionally all or part of a Notch ligand N-terminal domain; and iv) optionally one or more heterologous amino acid sequences; or a polynucleotide which codes for such a Notch ligand protein or polypeptide.

27. A Notch ligand protein or polypeptide which consists essentially of the following components: i) a Notch ligand DSL domain; ii) two Notch ligand EGF domains; iii) optionally all or part of a Notch ligand N-terminal domain; and iv) optionally one or more heterologous amino acid sequences; or a polynucleotide which codes for such a Notch ligand protein or polypeptide.

28. A Notch ligand protein or polypeptide which consists essentially of the following components: i) a Notch ligand DSL domain; ii) three Notch ligand EGF domains; iii) optionally all or part of a Notch ligand N-terminal domain; and iv) optionally one or more heterologous amino acid sequences; or a polynucleotide sequence which codes for such a Notch ligand protein or polypeptide.

29. A Notch ligand protein or polypeptide consisting essentially of the following components: i) a Notch ligand DSL domain; ii) 1 to 5 EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; for use in the treatment of disease.

30. A Notch ligand protein or polypeptide comprising: i) a Notch ligand DSL domain; ii) 1 to 5 (but no more than 5) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; for use in the treatment of disease.

31. A Notch ligand protein or polypeptide consisting essentially of the following components: i) a Notch ligand DSL domain; ii) 1 to 4 EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; for use in the treatment of disease.

32. A Notch ligand protein or polypeptide comprising: i) a Notch ligand DSL domain; ii) 1 to 4 (but no more than 4) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; for use in the treatment of disease.

33. A Notch ligand protein or polypeptide comprising: i) a Notch ligand DSL domain; ii) 1 (but no more than 1) EGF repeat domain; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; for use in the treatment of disease.

34. A Notch ligand protein or polypeptide comprising: i) a Notch ligand DSL domain; ii) 2 (but no more than 2 ) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; for use in the treatment of disease.

35. A Notch ligand protein or polypeptide comprising: i) a Notch ligand DSL domain; ii) 3 (but no more than 3) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; for use in the treatment of disease.

36. A Notch ligand protein or polypeptide comprising: i) a Notch ligand DSL domain; ii) 4 (but no more than 4) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; for use in the treatment of disease.

37. A Notch ligand protein or polypeptide comprising: i) a Notch ligand DSL domain; ii) 5 (but no more than 5) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide; for use in the treatment of disease.

38. A method of therapeutically modulating Notch signalling by administering a Notch ligand protein or polypeptide consisting essentially of the following components: i) a Notch ligand DSL domain; ii) 1 to 5 EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide.

39. A method of therapeutically modulating Notch signalling by administering a Notch ligand protein or polypeptide comprising: i) a Notch ligand DSL domain; ii) 1 to 5 (but no more than 5) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide.

40. A method of therapeutically modulating Notch signalling by administering a Notch ligand protein or polypeptide consisting essentially of the following components: i) a Notch ligand DSL domain; ii) 1 to 4 EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide.

41. A method of therapeutically modulating Notch signalling by administering a Notch ligand protein or polypeptide comprising: i) a Notch ligand DSL domain; ii) 1 to 4 (but no more than 4) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide.

42. A method of therapeutically modulating Notch signalling by administering a Notch ligand protein or polypeptide comprising: i) a Notch ligand DSL domain; ii) 1 (but no more than 1) EGF repeat domain; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide.

43. A method of therapeutically modulating Notch signalling by administering a Notch ligand protein or polypeptide comprising: i) a Notch ligand DSL domain; ii) 2 (but no more than 2) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide.

44. A method of therapeutically modulating Notch signalling by administering a Notch ligand protein or polypeptide comprising: i) a Notch ligand DSL domain; ii) 3 (but no more than 3) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide.

45. A method of therapeutically modulating Notch signalling by administering a Notch ligand protein or polypeptide comprising: i) a Notch ligand DSL domain; ii) 4 (but no more than 4) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide.

46. A method of therapeutically modulating Notch signalling by administering a Notch ligand protein or polypeptide comprising: i) a Notch ligand DSL domain; ii) 5 (but no more than 5) EGF repeat domains; iii) optionally all or part of a Notch ligand N-terminal domain; and iv) optionally one or more heterologous amino acid sequences; or a multimer of such a protein or polypeptide (wherein each monomer may be the same or different); or a polynucleotide coding for such a Notch ligand protein or polypeptide.

47. A vector comprising a polynucleotide coding for a Notch ligand protein or polypeptide as described in any one of paragraphs 25 to 37.

48. A host cell transformed or transfected with a vector as described in paragraph 47.

49. A cell displaying a Notch ligand protein or polypeptide as described in any one of paragraphs 25 to 37 on its surface or transfected with a polynucleotide coding for such a protein or polypeptide.

### REFERENCES (INCORPORATED HEREIN BY REFERENCE)

Altman J D et al Science 1996 274: 94-6.
Artavanis-Tsakonas S, et al. (1995) Science 268:225-232.
Artavanis-Tsakonas S, et al. (1999) Science 284:770-776.
Brucker K, et al. (2000) Nature 406:411-415.
Camilli et al. (1994) Proc Natl Acad Sci USA 91:2634-2638.
Chee M. et al. (1996) Science 274:601-614.
Hemmati-Brivanlou and Melton (1997) Cell 88:13-17.
Hicks C, et al. (2000) Nat. Cell. Biol. 2:515-520.
Iemura et al. (1998) PNAS 95:9337-9345.
Irvine K D (1999) Curr. Opin. Genet. Devel. 9:434-441.
Ju B J, et al. (2000) Nature 405:191-195.
Leimeister C. et al. (1999) Mech Dev 85(1-2):173-7.
Li et al. (1998) Immunity 8(1):43-55.
Lieber, T. et al. (1993) Genes Dev 7(10):1949-65.
Lu, F. M. et al. (1996) Proc Natl Acad Sci 93(11):5663-7.
Matsuno K, et al. (1998) Nat. Genet. 19:74-78.
Matsuno, K. et al. (1995) Development 121(8):2633-44.
Medhzhitov et al. (1997) Nature 388:394-397.
Meuer S. et al (2000) Rapid Cycle Real-time PCR, Springer-Verlag Berlin and Heidelberg GmbH&Co.
Moloney DJ, et al. (2000) Nature 406:369-375.
Munro S, Freeman M. (2000) Curr. Biol. 10:813-820.
Ordentlich et al. (1998) Mol. Cell. Biol. 18:2230-2239.
Osborne B, Miele L. (1999) Immunity 11:653-663.
Panin V M, et al. (1997) Nature 387:908-912.
Sasai et al. (1994) Cell 79:779-790.
Schroeter E H, et al. (1998) Nature 393:382-386.
Schroeter, E. H. et al. (1998) Nature 393(6683):382-6.
Struhl G, Adachi A. (1998) Cell 93:649-660.
Struhl, G. et al. (1998) Cell 93(4):649-60.
Takebayashi K. et al. (1994) J Biol Chem 269(7):150-6.
Tamura K, et al. (1995) Curr. Biol. 5:1416-1423.
Valenzuela et al. (1995) J. Neurosci. 15:6077-6084.
Weinmaster G. (2000) Curr. Opin. Genet. Dev. 10:363-369.
Wilson and Hemmati-Brivanlou (1997) Neuron 18:699-710.
Zhao et al. (1995) J. Immunol. 155:3904-3911.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A Notch ligand protein or polypeptide comprising:
i) a Notch ligand DSL domain; and
ii) 1 to 5 (but no more than 5) Notch ligand EGF repeat domains; and
iii) optionally all or part of a Notch ligand N-terminal domain; and
iv) optionally one or more heterologous amino acid sequences;
or a homogeneous or heterogeneous multimer of the protein or polypeptide;
or a polynucleotide encoding the Notch ligand protein or polypeptide.

2. The Notch ligand protein or polypeptide according to claim 1, comprising 1 to 4 (but no more than 4) Notch ligand EGF repeat domains.

3. The Notch ligand protein or polypeptide according to claim 1, wherein there is one Notch ligand EGF repeat domain.

4. The Notch ligand protein or polypeptide according to claim 1, wherein there are two Notch ligand EGF repeat domains.

5. The Notch ligand protein or polypeptide according to claim 1, wherein there are three Notch ligand EGF repeat domains.

6. The Notch ligand protein or polypeptide according to claim 1, wherein there are four Notch ligand EGF repeat domains.

7. The Notch ligand protein or polypeptide according to claim 1, wherein there are five Notch ligand EGF repeat domains.

8. The Notch ligand protein or polypeptide according to claim 1, wherein the Notch ligand protein or polypeptide activates a human Notch receptor.

9. The Notch ligand protein or polypeptide according to claim 1, wherein the heterologous amino acid sequence comprises all or part of an immunoglobulin F_{c} domain.

10. The Notch ligand protein or polypeptide according to claim 1, wherein the Notch ligand protein, polypeptide or polynucleotide comprises or encodes at least part of a mammalian Notch ligand.

11. The Notch ligand protein or polypeptide according to claim 1, wherein the Notch ligand protein, polypeptide or polynucleotide comprises or encodes at least part of a human Notch ligand

12. The Notch ligand protein or polypeptide according to claim 1, wherein the Notch ligand protein, polypeptide or polynucleotide comprises or encodes Notch ligand domains from Delta, Serrate or Jagged or domains having at least 30% amino acid sequence similarity thereto.

13. The Notch ligand protein or polypeptide according to claim 1, wherein the Notch ligand protein, polypeptide or polynucleotide comprises or encodes Notch ligand domains from Delta1, Delta 3, Delta 4, Jagged 1 or Jagged 2 or domains having at least 30% amino acid sequence similarity thereto.

14. A multimeric Notch ligand protein or polypeptide comprising monomers comprising:
i) a Notch ligand DSL domain; and
ii) 1 to 5 (but no more than 5) Notch ligand EGF repeat domains; and
iii) optionally all or part of a Notch ligand N-terminal domain; and
iv) optionally one or more heterologous amino acid sequences;
wherein the multimeric Notch ligand protein or polypeptide is homogeneous or heterogeneous.

15. The multimeric Notch ligand protein or polypeptide according to claim 14, wherein the monomers comprise 1 to 4 (but no more than 4) Notch ligand EGF repeat domains.

16. A composition comprising the Notch ligand protein or polypeptide according to claim 1, in combination with a pharmaceutically acceptable carrier.

17. A method of modulating Notch signalling in a subject by administering to the subject the Notch ligand protein or polypeptide according to claim 1.

18. A vector comprising a polynucleotide encoding the Notch ligand protein or polypeptide according to claim 1.

19. A host cell transformed or transfected with the vector as claimed in claim 18.

20. A cell displaying the Notch ligand protein or polypeptide according to claim 1 on its surface or a cell transfected with a polynucleotide encoding the Notch ligand protein or polypeptide according to claim 1.


SEQUENCE LISTING (51 sequences)

SEQ ID NO: 1 - protein, 63 aa, Drosophila melanogaster
WKTNKSESQYTSLEYDFRVTCDLNYYGSGCAKFCRPRDDSFGHSTCSETGEIICLTGWQGDYC

SEQ ID NO: 2 - protein, 63 aa, Homo sapiens
WSQDLHSSGRTDLKYSYRFVCDEHYYGEGCSVFCRPRDDAFGHFTCGERGEKVCNPGWKGPYC

SEQ ID NO: 3 - protein, 63 aa, Mus musculus
WSQDLHSSGRTDLRYSYRFVCDEHYYGEGCSVFCRPRDDAFGHFTCGDRGEKMCDPGWKGQYC

SEQ ID NO: 4 - protein, 63 aa, Rattus norvegicus
WSQDLHSSGRTDLRYSYRFVCDEHYYGEGCSVFCRPRDDAFGHFTCGERGEKMCDPGWKGQYC

SEQ ID NO: 5 - protein, 63 aa, Mus musculus
WRTDEQNDTLTRLSYSYRVICSDNYYGESCSRLCKKRDDHFGHYECQPDGSLSCLPGWTGKYC

SEQ ID NO: 6 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 7 - protein, 63 aa, Rattus norvegicus
WQTLKQNTGIAHFEYQIRVTCDDHYYGFGCNKFCRPRDDFFGHYACDQNGNKTCMEGWMGPEC

SEQ ID NO: 8 - protein, 63 aa, Mus musculus
WQTLKQNTGIAHFEYQIRVTCDDHYYGFGCNKFCRPRDDFFGHYACDQNGNKTCMEGWMGPDC

SEQ ID NO: 9 - protein, 63 aa, Homo sapiens
WQTLKQNTGVAHFEYQIRVTCDDYYYGFGCNKFCRPRDDFFGHYACDQNGNKTCMEGWMGREC

SEQ ID NO: 10 - protein, 63 aa, Gallus gallus
WQTLKHNTGAAHFEYQIRVTCAEHYYGFGCNKFCRPRDDFFTHHTCDQNGNKTCLEGWTGPEC

SEQ ID NO: 11 - protein, 63 aa, Gallus gallus
WKTLQFNGPVANFEVQIRVKCDENYYSALCNKFCGPRDDFVGHYTCDQNGNKACMEGWMGEEC

SEQ ID NO: 12 - protein, 63 aa, Mus musculus
WKSLHFSGHVAHLELQIRVRCDENYYSATCNKFCRPRNDFFGHYTCDQYGNKACMDGWMGKEC

SEQ ID NO: 13 - protein, 63 aa, Homo sapiens; identical to SEQ ID NO: 12

SEQ ID NO: 14 - protein, 63 aa, Rattus norvegicus; identical to SEQ ID NO: 12

SEQ ID NO: 15 - protein, 63 aa, Homo sapiens; identical to SEQ ID NO: 12

SEQ ID NO: 16 - protein, 63 aa, Drosophila melanogaster
WKTLDHIGRNARITYRVRVQCAVTYYNTTCTTFCRPRDDQFGHYACGSEGQKLCLNGWQGVNC

SEQ ID NO: 17 - protein, 723 aa, Homo sapiens [sequence omitted]

SEQ ID NO: 18 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 19 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 20 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 21 - protein, 1238 aa, Homo sapiens [sequence omitted]

SEQ ID NO: 22 - protein, 43 aa, Artificial Sequence
  Description of Artificial Sequence Synthetic consensus sequence
CXXXXXXXXCXXXCXXXXXXXXXXXCXXXXXXXCXXXXXXXXC

SEQ ID NO: 23 - protein, 43 aa, Artificial Sequence; identical to SEQ ID NO: 22
  Description of Artificial Sequence Synthetic consensus sequence

SEQ ID NO: 24 - protein, 43 aa, Artificial Sequence
  Description of Artificial Sequence Synthetic consensus sequence
CXXXYYXXXCXXXCRPRXDXFGHXXCXXXGXXXCXXGWXGXXC

SEQ ID NO: 25 - protein, 175 aa, Artificial Sequence
  Description of Artificial Sequence Synthetic EGF-like domain
XXXXCXXXXXXXXXXXXXXXXXXXXXXXXXXXXXXXXXXXXXXXXXXXXXXXXCXXXXXXXXXXXXCXXXXXXXXXXXXXXXXXXXXXXXXXXXXXXXXXXXXXXXXXXXXXXXXXXXXXXXXXXXXXXXXXXXXXXCXXXXXXCXXGXXXXXXXXXXXXXXXXXXXXXXGXXCX

SEQ ID NO: 26 - DNA, 26 bp, Artificial Sequence
  Description of Artificial Sequence Synthetic primer
caccatgggcagtcggtgcgcgctgg

SEQ ID NO: 27 - DNA, 22 bp, Artificial Sequence
  Description of Artificial Sequence Synthetic primer
gtagttcaggtcctggttgcag

SEQ ID NO: 28 - DNA, 26 bp, Artificial Sequence
  Description of Artificial Sequence Synthetic primer
caccatgggcagtcggtgcgcgctgg

SEQ ID NO: 29 - DNA, 45 bp, Artificial Sequence
  Description of Artificial Sequence Synthetic primer
ggatatgggcccttggtggaagcgtagttcaggtcctggttgcag

SEQ ID NO: 30 - protein, 291 aa, Artificial Sequence
  Description of Artificial Sequence Synthetic protein construct
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNY

SEQ ID NO: 31 - DNA, 26 bp, Artificial Sequence
  Description of Artificial Sequence Synthetic primer
caccatgggcagtcggtgcgcgctgg

SEQ ID NO: 32 - DNA, 45 bp, Artificial Sequence
  Description of Artificial Sequence Synthetic primer
ggatatgggcccttggtggaagcctcgtcaatccccagctcgcag

SEQ ID NO: 33 - protein, 331 aa, Artificial Sequence
  Description of Artificial Sequence Synthetic protein construct
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDE

SEQ ID NO: 34 - DNA, 26 bp, Artificial Sequence
  Description of Artificial Sequence Synthetic primer
caccatgggcagtcggtgcgcgctgg

SEQ ID NO: 35 - DNA, 22 bp, Artificial Sequence
  Description of Artificial Sequence Synthetic primer
ggtcatggcactcaattcacag

SEQ ID NO: 36 - DNA, 26 bp, Artificial Sequence
  Description of Artificial Sequence Synthetic primer
caccatgggcagtcggtgcgcgctgg

SEQ ID NO: 37 - DNA, 45 bp, Artificial Sequence
  Description of Artificial Sequence Synthetic primer
ggatatgggcccttggtggaagcggtcatggcactcaattcacag

SEQ ID NO: 38 - protein, 369 aa, Artificial Sequence
  Description of Artificial Sequence Synthetic protein construct
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMT

SEQ ID NO: 39 - DNA, 26 bp, Artificial Sequence
  Description of Artificial Sequence Synthetic primer
caccatgggcagtcggtgcgcgctgg

SEQ ID NO: 40 - DNA, 25 bp, Artificial Sequence
  Description of Artificial Sequence Synthetic primer
cctgctgacgggggcactgcagttc

SEQ ID NO: 41 - DNA, 26 bp, Artificial Sequence
  Description of Artificial Sequence Synthetic primer
caccatgggcagtcggtgcgcgctgg

SEQ ID NO: 42 - DNA, 48 bp, Artificial Sequence
  Description of Artificial Sequence Synthetic primer
ggatatgggcccttggtggaagccctgctgacgggggcactgcagttc

SEQ ID NO: 43 - protein, 484 aa, Artificial Sequence
  Description of Artificial Sequence Synthetic protein construct
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSR

SEQ ID NO: 44 - DNA, 26 bp, Artificial Sequence
  Description of Artificial Sequence Synthetic nucleotide sequence
gatctggggggctataaaagggggta

SEQ ID NO: 45 - DNA, 26 bp, Artificial Sequence
  Description of Artificial Sequence Synthetic nucleotide sequence
agcttacccccttttatagcccccca

SEQ ID NO: 46 - DNA, 50 bp, Artificial Sequence
  Description of Artificial Sequence Synthetic nucleotide sequence
gatcccgactcgtgggaaaatgggcggaagggcaccgtgggaaaatagta

SEQ ID NO: 47 - DNA, 50 bp, Artificial Sequence
  Description of Artificial Sequence Synthetic nucleotide sequence
gatctactattttcccacggtgcccttccgcccattttcccacgagtcgg

SEQ ID NO: 48 - DNA, 26 bp, Artificial Sequence
  Description of Artificial Sequence Synthetic primer
caccatgggcagtcggtgcgcgctgg

SEQ ID NO: 49 - DNA, 45 bp, Artificial Sequence
  Description of Artificial Sequence Synthetic primer
gtctacgtttaaacttaacactcgtcaatccccagctcgcaggtg

SEQ ID NO: 50 - DNA, 999 bp, Artificial Sequence
  Description of Artificial Sequence Synthetic D1E3Cys coding sequence
atgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgttaa

SEQ ID NO: 51 - protein, 332 aa, Artificial Sequence
  Description of Artificial Sequence Synthetic D1E3Cys protein
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDEC